(19) United States
(12) Patent Application Publication
(10) Pub. No.: US 2018/0271792 A1
(43) Pub. Date: Sep. 27, 2018

(54) ORAL DELIVERY OF PHYSIOLOGICALLY ACTIVE SUBSTANCES

(71) Applicant: Rezolute, Inc., Louisville, CO (US)

(72) Inventors: Sankaram Mantripragada, Windsor, CO (US); Luke Amer, Louisville, CO (US); Kathleen M. Campbell, Firestone, CO (US); Xueyan Wang, Foster City, CA (US)

(73) Assignee: Rezolute, Inc., Louisville, CO (US)

(21) Appl. No.: 15/922,651

(22) Filed: Mar. 15, 2018

Related U.S. Application Data
(60) Provisional application No. 62/475,624, filed on Mar. 23, 2017.

(51) Int. Cl. A61K 9/48; A61K 47/60; A61K 38/28

(52) U.S. Cl. CPC A61K 9/4866; A61K 9/4875; A61K 38/28; A61K 47/60; A61K 9/4858

(57) ABSTRACT

Embodiments may include a composition for oral drug delivery. The composition may include a physiologically active substance, a carrier compound, a mucoadhesive compound, and a permeation enhancer. The physiologically active substance may be transported across the stomach. The physiologically active substance may be stable and not degrade in the harsh gastric acid environment. To help protect the physiologically active substance, the physiologically active substance is mixed with the carrier. The carrier may be a liquid insoluble in the gastric acid of the stomach. The physiologically active substance may be soluble in the carrier. The mucoadhesive compound may be used to promote adsorption of the physiologically active substance to the lining of the stomach. The permeation enhancer may facilitate the transport of the physiologically active substance across the wall of the stomach.

### CROSS-REFERENCE TO RELATED APPLICATION

The present application claims the benefit of and priority of U.S. Provisional Application No. 62/475,624, filed Mar. 23, 2017, the entire contents of which are hereby incorporated by reference for all purposes.

### BACKGROUND

Delivery of physiologically active substances such as, small molecule drugs, hormones, proteins, diagnostics, and other medically active substances into a patient faces a number of challenges. The physiologically active substance has to be delivered into the patient. One way to deliver the physiologically active substance is by injection. Injection may allow the physiologically active substance to reach the bloodstream or targeted area for treatment quickly or directly, but injection may be inconvenient or painful for the patient. Many physiologically active substances have to be administered frequently, including several times a day. A more frequent administration schedule may increase the inconvenience to the patient, may decrease the compliance rate by patients, and may lead to less than optimal outcomes for the patient. If the physiologically active substance is administered by injection, another injection increases the frequency of pain, the risk of infection, and the probability of an immune response in the patient. An alternative to injection is ingestion. Ingestion is often more convenient and less intrusive than injection. However, with ingestion, the physiologically active substance may have to pass through a patient's digestive system and may degrade before reaching the bloodstream or targeted area for treatment. As a result, injection is often used instead of ingestion. For example, treatment for diabetes typically requires insulin injections and not oral delivery of insulin. There remains a need to reliably orally deliver physiologically active substances to the bloodstream or targeted area for treatment. The methods and compositions described herein provide solutions to these and other needs.

### BRIEF SUMMARY

Embodiments of the present technology allow for the oral delivery of physiologically active substances to the bloodstream of a human or other animal. The physiologically active substances are transported mainly across the wall of the stomach. In order for the physiologically active substance to be transported across the stomach before degrading in the harsh environment, the physiologically active substance is mixed with a carrier. The carrier may be a liquid insoluble in the gastric acid of the stomach. The physiologically active substance may be soluble in the carrier. The carrier may protect the physiologically active substance from the gastric acid and pepsin in the stomach. A mucoadhesive compound may be used to promote adsorption of the physiologically active substance to the lining of the stomach. A permeation or absorption enhancer may facilitate the transport of the physiologically active substance across the wall of the stomach. The oral delivery of the physiologically active substance may not need certain coatings or inhibitors, which may have undesirable side effects.

Embodiments may include a composition for oral drug delivery. The composition may include a physiologically active substance, a carrier compound, a mucoadhesive compound, and a permeation enhancer.

Embodiments may include a drug formulation for oral delivery. The drug formulation may include a physiologically active substance. The drug formulation may also include a material that includes at least one of a mucoadhesive compound, a permeation enhancer, an inverted micelle, or a compound in which the physiologically active substance forms an inclusion complex. The physiologically active substance compound may include the center of mass of the drug formulation. The material may be in contact with the physiologically active substance. A portion of the material may be disposed farther from the center of mass than any portion of the physiologically active substance.

Embodiments may include a method of manufacturing a drug for the oral delivery of a physiologically active substance. The method may include combining a physiologically active substance, a carrier compound, a mucoadhesive compound, and a permeation enhancer. The method may further include encapsulating the physiologically active substance, the carrier compound, the mucoadhesive compound, and the permeation enhancer in a capsule. The capsule may be configured to dissolve in gastric acid to release the physiologically active substance, the carrier compound, the mucoadhesive compound, and the permeation enhancer. The capsule may be coated with a mucoadhesive compound.

Embodiments may also include a method of treatment. Methods may include orally administering to a person a capsule containing a composition. The composition may include a physiologically active substance, a carrier compound, a mucoadhesive compound, and a permeation enhancer. The methods may also include dissolving a portion of the capsule in a stomach of the person to release the physiologically active substance and the carrier compound into the stomach. Methods may further include adsorbing a portion of the physiologically active substance onto a wall of the stomach. In addition, methods may include transporting the physiologically active substance across the wall of the stomach into a bloodstream.

### BRIEF DESCRIPTION OF THE DRAWINGS

FIG. 1 shows an illustration of the oral delivery of a capsule containing a physiologically active substance according to embodiments of the present technology.

FIGS. 2A-2E show illustrations of the transport processes involved in oral delivery of a physiologically active substance according to embodiments of the present technology.

FIGS. 3A-3G show illustrations of the structural layers of the oral delivery composition according to embodiments of the present technology.

FIG. 4 shows a method of manufacturing a drug for the oral delivery of a physiologically active substance according to embodiments of the present technology.

FIG. 5 shows a method of treatment according to embodiments of the present technology.

### DETAILED DESCRIPTION

Conventional methods of administering a physiologically active substance include injection. More recent efforts have focused on developing oral methods to administer a physiologically active substance. However, most efforts focus on protecting the physiologically active substance in the gastric acid of the stomach until the physiologically active substance reaches the small intestine. The physiologically active substance is then delivered to the bloodstream through the small intestine. In order for the physiologically active substance to not fully degrade in the stomach, prior efforts include adding an enteric coating and/or a protease inhibitor. An enteric coating is a coating resistant to acid hydrolysis. A protease inhibitor may also include a peptidase inhibitor. The enteric coating and the protease inhibitor may interfere with normal digestion of food and may have side effects of bloating and constipation. Additionally, for a significant amount of physiologically active substance to be absorbed through the small intestine, a high concentration of the physiologically active substance may need to be in the composition before ingestion. Passing a physiologically active substance through the small intestine where there are not usually the appropriate receptors for the physiologically active substance may lead to negative outcomes. For example, insulin receptors are not typically located near the small intestine, but instead near the pancreas and liver. An insulin protein passing through the intestinal wall does not have a direct path to the receptors in the pancreas and liver. Instead, the insulin-like growth factor (IGF) receptors. An increased level of insulin binding to IGF receptors leads to mitogenesis and has been linked to cancer.

Embodiments of the present technology may allow for improved oral delivery of physiologically active substances. Instead of delivering the physiologically active substance across the intestinal wall, the physiologically active substance may be delivered across the stomach wall. Transporting the physiologically active substance across the stomach wall may include several advantages. The pancreas or liver may include receptors for the protein or peptide, and transport across the stomach wall may provide a direct or reduced path to the receptors compared to transport across the intestinal wall. Because the physiologically active substance does not need to reach the intestine, the physiologically active substance may not include an enteric coating to protect the physiologically active substance. The coatings may have undesirable side effects. The physiologically active substance concentration before ingestion may not need to be as high in a path across the stomach wall instead of across the intestinal wall because more of the physiologically active substance may not degrade through a shorter time in the digestive tract or because more of the physiologically active substance may be absorbed across the stomach wall than the intestinal wall. The lack of enteric coatings may decrease the cost to administer the physiologically active substance.

In order for the physiologically active substance to be transported across the stomach, the physiologically active substance should be stable and not degrade in the harsh gastric acid environment, and the physiologically active substance should be absorbed through the stomach wall. To this end, embodiments of the present technology include methods of increasing stability of the physiologically active substance in the stomach and enhancing absorption of the physiologically active substance. To help protect the physiologically active substance, the physiologically active substance is mixed with a carrier. The carrier is a liquid insoluble in the gastric acid of the stomach. The physiologically active substance may be soluble in the carrier. A mucoadhesive compound may be used to promote adsorption of the physiologically active substance to the lining of the stomach. A permeation enhancer may facilitate the transport of the physiologically active substance across the wall of the stomach.

Physiologically active substance means a natural, synthetic, or genetically engineered chemical or biological compound that is known in the art as modulating physiological processes in order to afford diagnosis of, prophylaxis against, or treatment of an undesired existing condition in a living being. Physiologically active substances include drugs such as antianginas, antiarrhythmics, antiasthmatic substances, antibiotics, antidiabetics, antifungals, antihistamines, antihypertensives, antiparasitics, antineoplastics, antitumor drugs, antivirals, cardiac glycosides, herbicides, hormones, immunomodulators, monoclonal antibodies, neurotransmitters, nucleic acids, proteins, radio contrast substances, radionuclides, sedatives, analgesics, steroids, tranquilizers, vaccines, vasopressors, anesthetics, peptides, small molecules, and the like.

Prodrugs, which undergo conversion to the indicated physiologically active substances upon local interactions with the intracellular medium, cells, or tissues, can also be employed in place of or in addition to the physiologically active substance in embodiments. Any acceptable salt of a particular physiologically active substance, which is capable of forming such a salt, is also envisioned as being included in place of or in addition to the physiologically active substance in embodiments. Salts may include halide salts, phosphate salts, acetate salts, organic acid salts, and other salts.

The physiologically active substances may be used alone or in combination. The amount of the substance in the pharmaceutical composition may be sufficient to enable the diagnosis of, prophylaxis against, or the treatment of an undesired existing condition in a living being. Generally, the dosage may vary with the age, condition, sex, and extent of the undesired condition in the patient, and can be determined by one skilled in the art. The dosage range appropriate for human use includes a range of 0.1 to 6,000 mg of the physiologically active substance per square meter of body surface area.

Physiologically active substances may include proteins or peptides. Proteins or peptides may include insulin, human growth hormone, glucagon-like peptide-1, parathyroid hormone, a fragment of parathyroid hormone, enfuvirtide, or octreotide.

Insulin is normally produced by the pancreas. Insulin regulates the metabolism of glucose in the blood. A high level of glucose or other high blood sugar may be an indication of a disorder in the production of insulin and may be an indication of diabetes. Insulin is often administered by injection as a treatment for diabetes.

Another protein that may be used as a physiologically active substance is glucagon-like peptide-1 (GLP-1). GLP-1, a 31 amino acid peptide, is an incretin, a hormone that can decrease blood glucose levels. GLP-1 may affect blood glucose by stimulating insulin release and inhibiting glucagon release. GLP-1 also may slow the rate of absorption of nutrients into the bloodstream by reducing gastric emptying and may directly reduce food intake. The ability of GLP-1 to affect glucose levels has made GLP-1 a potential treatment for type 2 diabetes and other afflictions. In its unaltered state, GLP-1 has an in vivo half-life of less than two minutes as a result of proteolysis.

Proteins or peptides may include human growth hormone. Human growth hormone (hGH), a 191 amino acid peptide, is a hormone that increases cell growth and regeneration. hGH may be used to treat growth disorders and deficiencies. For instance, hGH may be used to treat short stature in children or growth hormone deficiencies in adults. Conventional methods of administering hGH include daily subcutaneous injection.

Similar to hGH and GLP-1, enfuvirtide (Fuzeon®) is a physiologically active substance that may face challenges when administered to patients. Enfuvirtide may help treat HIV and AIDS. However, enfuvirtide may have to be injected subcutaneously twice a day. Injections may result in skin sensitivity reaction side effects, which may discourage patients from continuing use of enfuvirtide. An oral enfuvirtide treatment may be needed to increase patient compliance, lower cost, and enhance the quality of life for patients with HIV and AIDS.

Another physiologically active substance is parathyroid hormone (PTH) or a fragment of PTH. PTH is an anabolic (bone forming) substance. PTH may be secreted by the parathyroid glands as a polypeptide containing 84 amino acids with a molecular weight of 9,425 Da. The first 34 amino acids may be the biologically active moiety of mineral homeostasis. A synthetic, truncated version of PTH is marketed by Eli Lilly and Company as Forteo® Teriparatide. PTH or a fragment of PTH may be used to treat osteoporosis and hypoparathyroidism. Teriparatide may often be used after other treatments as a result of its high cost and required daily injections. As with other physiologically active substances, an oral PTH treatment may be desired.

The physiologically active substance may include a small molecule. Small molecules may include drugs defined by the Biopharmaceutics Classification System (BCS), which is a system to classify orally delivered drugs based on their aqueous solubility and intestinal permeability. BCS classifies orally delivered drug substances into four classes: Class 1, high permeability, high solubility; Class II, high permeability, low solubility; Class III, low permeability, high solubility; Class IV, low permeability, low solubility. The solubility classification is based on a United States Pharmacopoeia (USP); a drug substance is considered highly soluble when the highest strength is soluble in 250 mL or less of aqueous media within the pH range of 1-6.8 at 37±1° C. A drug substance is considered to be highly permeable when the systemic bioavailability is determined to be 85 percent or more of an administered dose based on a mass balance determination or in comparison to an intravenous reference dose. Additional information regarding small molecules may be found in Amidon G L, Lennernäs H, Shah V P, and Crison J R, 1995, A Theoretical Basis For a Biopharmaceutics Drug Classification: The Correlation of In Vitro Drug Product Dissolution and In Vivo Bioavailability, Pharm Res, 12: 413-420, the contents of which are incorporated herein by reference for all purposes.

Additional information on the proteins and conjugates of the proteins can be found in U.S. patent application Ser. No. 10/553,570, filed Apr. 8, 2004 (issued as U.S. Pat. No. 9,040,664 on May 26, 2015). Information regarding the concentration release profiles of proteins and conjugates can be found in U.S. patent application Ser. No. 14/954,701, filed Nov. 30, 2015. The contents of patent applications, publications, and all other references in this disclosure are incorporated herein by reference for all purposes.

### I. APPROACH

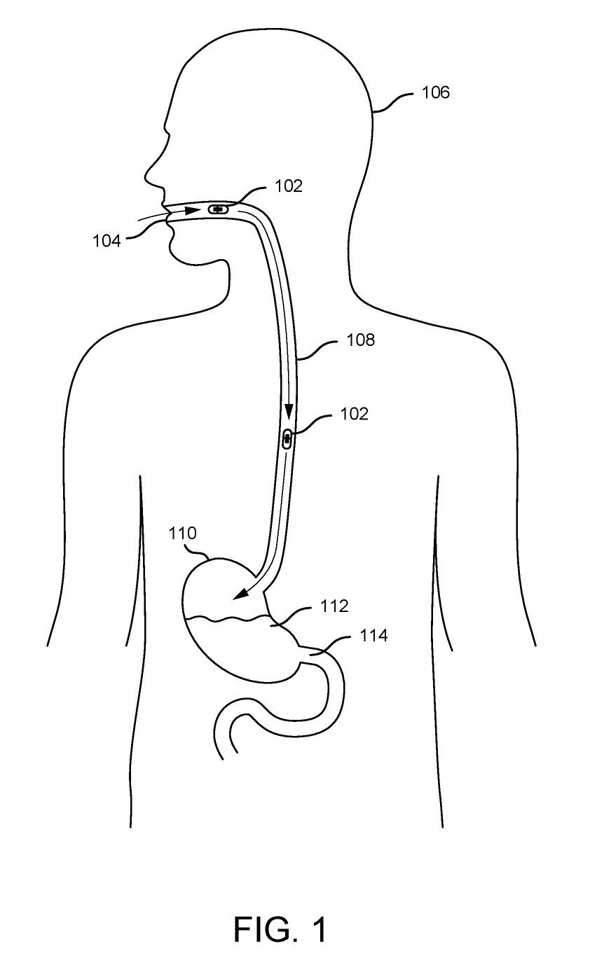

FIG. 1 shows an illustration of the oral delivery of a capsule 102 containing a physiologically active substance. Capsule 102 may be ingested through the mouth of a person 106. The capsule may travel down an esophagus 108 into a stomach 110. The stomach includes gastric fluid 112, which may also include pepsin enzyme. Capsule 102 may dissolve in stomach 110 and the physiologically active substance may be absorbed across the stomach wall. Capsule 102 may not travel to a duodenum 114 and the small intestine or other downstream parts of the digestive tract. FIG. 1 is provided for illustrative purposes, and the components are not drawn to scale.

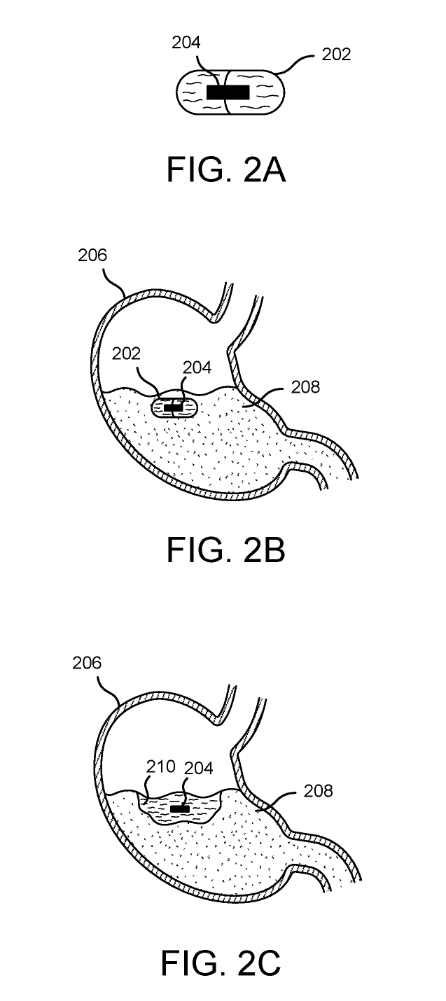

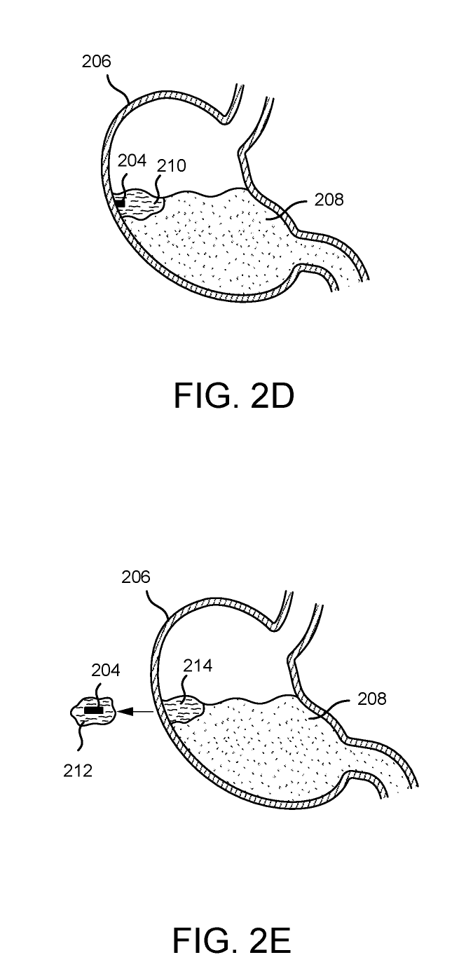

FIGS. 2A-2E show illustrations of the transport processes involved in oral delivery of a physiologically active substance. FIG. 2A shows an illustration of a capsule 202. Capsule 202 includes a physiologically active substance 204. Other compounds may also be included in capsule 202. For example, the other compounds may include a carrier compound, a mucoadhesive compound, and a permeation enhancer.

FIG. 2B shows capsule 202 in stomach 206. Stomach 206 contains a fluid 208, which includes gastric fluid and pepsin. Gastric fluid and pepsin may each individually degrade the physiologically active substance. Fluid 208 may dissolve capsule 202, which may release compounds in the capsule, including a physiologically active substance 204.

FIG. 2C shows physiologically active substance 204 in stomach 206 after capsule 202 has been dissolved. Physiologically active substance 204 is immersed in a carrier compound 210, which may serve to protect physiologically active substance 204 from fluid 208. Carrier compound 210 may be insoluble in fluid 208. For example, carrier compound 210 may be an organic phase, an oil phase, or a non-polar phase. Carrier compound 210 may include an oil. Physiologically active substance 204 may be partially or completely soluble in carrier compound 210. Carrier compound 204 may have a density less than water or fluid 208. As a result, carrier compound 210, along with the physiologically active substance 204, may float on top of fluid 208. Stomach 206 may normally never empty of fluid 208, and carrier compound 210 may float on top of fluid 208 for several hours.

FIG. 2D shows physiologically active substance 204 and carrier compound 210 migrating to a wall of stomach 206. The migration may be the result of normal fluid flows in the stomach. Physiologically active substance 204 may adsorb onto the stomach wall in order to prevent physiologically active substance 204 from migrating away from the stomach wall. A mucoadhesive substance, which may have been included in capsule 202, may aid in adsorption of the physiologically active substance 204 onto the stomach wall.

FIG. 2E shows physiologically active substance 204, along with a portion 212 of carrier compound, transported across the stomach wall. A portion 214 of carrier compound may remain in stomach 206. A permeation enhancer compound may aid the transport of physiologically active substance 204 across the cells of the stomach wall. Physiologically active substance 204 may then travel through the bloodstream to a receptor for the protein or peptide compound.

Some of physiologically active substance originally in capsule 202 may not be transported across the stomach wall. Some of the physiologically active substance may be lost to the gastric fluid or pepsin, despite the carrier compound and any other compounds that may help protect the physiologically active substance. Some of physiologically active substance may leave the carrier compound and enter the gastric fluid. The physiologically active substance may not be fully immersed in the carrier compound, and some of the physiologically active substance may become exposed to the gastric fluid. Additional losses may be incurred when not all the physiologically active substance is transported across the stomach wall. In addition, not all of the physiologically active substance transported across the stomach wall may reach receptors for the physiologically active substance. The initial dose of physiologically active substance in the capsule can be tailored to account for expected losses.

### II. COMPOSITIONS

Embodiments may include a composition for oral drug delivery. The composition may include a physiologically active substance, a carrier compound, a mucoadhesive compound, and a permeation enhancer.

The physiologically active substance may include any physiologically active substance described herein, including insulin, human growth hormone, glucagon-like peptide-1 (GLP-1), parathyroid hormone (PTH), a fragment of parathyroid hormone, enfuvirtide, or octreotide. Insulin, unless context indicates otherwise, refers to human insulin. The physiologically active substance may include a conjugate with PEG. For example, physiologically active substance may include an insulin-PEG conjugate or a GLP-1-PEG conjugate. The PEG may have a molecular weight in a range from 2 kDa to 5 kDa. PEGylated insulin may be referred to as peginsulin, PEG-insulin or insulin-PEG.

The physiologically active substance may include a protein or peptide analog, homolog, or derivative. Analogs are compounds that have one or several amino acids of the protein or peptide sequence, and either the rest of the sequence is replaced by a different amino acid or more amino acids are added to the sequence. For insulin, insulin analogs include insulin lispro, insulin aspart, insulin glulisine, and insulin glargine. Homologs are protein or peptide compounds from different animals. For example, dog insulin, pig insulin, and rat insulin are insulin homologs. In addition, insulin homologs may include mammal insulin, fish insulin, reptile insulin, and amphibian insulin. Derivatives are a protein or peptide compound, analog, or homolog with a moiety attached. For example, detemir, degludec, and PEG-insulin are insulin derivatives. The analogs, homologs, and derivatives should have a similar or same metabolic effect in an animal as the protein or peptide compound. For example, insulin analogs, insulin homologs, and insulin derivatives may have a metabolic effect on glucose in an animal.

Embodiments may include GLP-1, GLP-1 agonist, or a GLP-1 analog, homolog, or derivative. GLP-1 analogs and agonists include exendin, semaglutide, liraglutide, dulaglutide, albiglutide, and lixisenatide. GLP-1 homologs may include dog GLP-1, pig GLP-1, and rat GLP-1 are GLP-1 homologs. In addition, GLP-1 homologs may include mammal GLP-1, fish GLP-1, reptile GLP-1, and amphibian GLP-1. PEG-GLP-1 is an insulin derivative. GLP-1 analogs, GLP-1 homologs, and GLP-1 derivatives may respond to glucose by inducing a pancreas to release insulin.

Compositions may include any combination of protein or peptide compounds. For example, the composition may include any combination of insulin, insulin analog, insulin homolog, insulin derivative, GLP-1, GLP-1 analog, GLP-1 homolog, GLP-1 derivative, or PEGylated compounds thereof. For example, the composition may include an insulin, a GLP-1, a PEGylated insulin, and a PEGylated GLP-1.

The physiologically active substance may include a small molecule. Small molecules may include any small molecules described herein. Small molecules may include antipyretics, analgesics, antimalarial drugs, antibiotics, antiseptics, mood stabilizers, hormone replacements, oral contraceptives, stimulants, tranquilizers, and statins.

The carrier compound may be water insoluble. Gastric acid is an aqueous mixture, and a carrier compound should not mix with the gastric acid in order to slow degradation of the physiologically active substance compound. The carrier compound may include an amphipathic and water-immiscible compound. The carrier compound may include fish oil, docosahexaenoic acid (DHA), esterified triglycerides, omega fatty acids, olive oil, orange oil, krill oil, lemon oil, safflower oil, castor oil, hydrogenated oils, algal oils, or mixtures thereof. Fish oils may include oils from mackerel, herring, tuna, salmon, and cod liver. Carrier compounds may include whale blubber oil, seal blubber oil, bacon oil, lard, and liquefied butter. The carrier compound may also be a compound with a high bioavailability, a compound that can be absorbed into the bloodstream across the stomach wall. The carrier compound may be on the GRAS (generally regarded as safe) FDA registry. The carrier compound may be included at a ratio of 1 mL of carrier for every 1.5 mg of physiologically active substance equivalent. In some embodiments, the carrier compound may be added at a ratio of 0.1 to 0.5 mL, 0.5 mL to 1 mL, 1 mL to 1.5 mL, 1.5 mL to 2.0 mL, 2.0 mL to 2.5 mL, 2.5 mL to 3.0 mL, or greater than 3 mL for every 1.5 mg of physiologically active substance.

The mucoadhesive compound may include a cyclodextrin (e.g, Hepakis 2,6-B-O-methyl-B-cyclodextrin), a starch, a poly(d,l-lactide-co-glycolide) (PLGA), a caprolactone, or a food additive. Mucoadhesive compounds may include polymers derived from polyacrylic acid (e.g., polycarbophil, carbomers), polymers derived from cellulose (e.g., hydroxyethylcellulose, carboxymethylcellulose, hydroxypropylmethylcellulose), alginates, chitosan, lectins, ester groups of fatty acids (e.g., glyceryl monooleate, glyceryl monolinoleate), invasins, fimbrial proteins, antibodies, thiolated molecules (e.g., thiolated polymers), and derivatives thereof. Polymers used as mucoadhesive compounds may be cationic, anionic, or nonionic. Mucoadhesive compounds may include Polaxamer 188. Mucoadhesive compounds are described in Carvalho et al., “Mucoadhesive drug delivery systems,” Brazilian J. of Pharm. Sci., 45(1) (2010), the contents of which are incorporated herein by reference for all purposes.

Cyclodextrin may form an inclusion complex with the physiologically active substance or the cyclodextrin may form an inclusion complex with the PEG component of a PEGylated physiologically active substance. The cyclodextrin may include α-cyclodextrin, β-cyclodextrin, or γ-cyclodextrin. Cyclodextrin may also include chemically modified cyclodextrin, which may include hydroxypropyl-B-cyclodextrin, sulfobutyl ether B-cylcodextrin, randomly methylated B cyclodextrin, hydroxypropyl-gamma-cyclodextrin, polymerized cyclodextrins, epichlorohydrin-B-cyclodextrin, or carboxy methyl epichlorohydrin beta cyclodextrin.

Without intending to be bound by theory, it is speculated that the physiologically active substance may be protected in the cyclodextrin ring structure from degrading in gastric acid. An inclusion complex may be formed by any size PEG with any one of α-cyclodextrin, β-cyclodextrin, γ-cyclodextrin, or a chemically modified cyclodextrin. The inclusion complex may include one or more compounds associating with the physiologically active substance. For example, multiple cyclodextrin molecules may associate with a singular PEGylated protein. The inclusion complex may be formed with 0.5 molar to 1 molar excess, 1 molar to 2 molar excess, 2 molar to 3 molar excess, 3 molar to 4 molar excess, 4 molar to 5 molar excess, 5 molar to 10 molar excess, 10 molar to 15 molar excess, 15 molar to 20 molar excess, or greater than 20 molar excess.

The permeation enhancer may include a positively charged molecule, a negatively charged molecule, or a zwitterionic molecule. The permeation enhancer may include an amphiphilic molecule. The permeation enhancer may include a neutral molecule, such as alkyl glucoside. Positively charged molecules may include alkyl cholines, acyl cholines, and bile salts. Negatively charged molecules may include sodium dodecyl sulfate. Zwitterionic molecules may include phospholipids, sphingolipids, and dodecylphosphocholine (DPC). Permeation enhancers may include 1,2-dipalmitoyl-sn-glycerol-3-phosphoglycerol (DPPG), 1-palmitoyl-2-oleoyl-sn-glycero-3-phosphoethanolamine (POPE), deoxycholic acid, sodium deoxycholate, sodium glycocholate, taurocholic acid sodium salt, ethylenediaminetetraacetic acid (EDTA), N-dodecyl B-D-maltoside, tridecyl B-D-maltoside, sodium dodecyl sulfate (SDS), sodium docusate (DSS), bile salts, nano emulsions (e.g., droplet size of less than 150 nm, based on Pluronic® copolymers), cyclodextrin, chitosan derivatives (e.g., protonated chitosan, trimethyl chitosan chloride), saponins, and straight chain fatty acids (e.g., capric acid, lauric acid, oleic acid). Permeation enhancers may include Polaxamer 188. Permeation enhancers are described in Shaikh et al., “Permeability enhancement techniques for poorly permeable drugs: A review,” J. of Appl. Pharm. Sci., 02(06) (2012), the contents of which are incorporated herein by reference for all purposes.

The permeation enhancer may be included at a ratio of 3 mg per 1.5 mg of physiologically active substance. In some embodiments, the permeation enhancer may be included at a ratio from 0.5 mg to 1.0 mg, 1.0 mg to 1.5 mg, 1.5 mg to 2.0 mg, 2.0 mg to 2.5 mg, 2.5 mg to 3.0 mg, 3.0 mg to 3.5 mg, 3.5 mg to 4.0 mg, or greater than 4.0 mg for every 1.5 mg of physiologically active substance.

The composition may also include a capsule encapsulating the physiologically active substance, the carrier compound, the mucoadhesive compound, and the permeation enhancer. The capsule may be configured to degrade in a stomach. In other words, the capsule may be configured such that at least a portion of the capsule degrades or dissolves away in the stomach so as to release the contents of the capsule. In some cases, the entire capsule may degrade away or dissolve away in the stomach. The capsule materials may include gelatin, polysaccharides, and plasticizers. The capsule material may include an enteric coating.

The composition may also include a hydrophobic anion of an organic acid. The hydrophobic anion of an organic acid may increase the hydrophobicity of the physiologically active substance, which may allow the physiologically active substance to stay in the carrier compound for a longer duration. The organic acid may include pamoic acid, docusate (DSS), furoic acid, or mixtures thereof. The hydrophobic anion may include a pamoate anion, a docusate anion, or a furoate anion. In these or other examples, the hydrophobic anion may be a fatty acid anion, a phospholipid anion, a polystyrene sulfonate anion, or mixtures thereof. The phospholipid of the phospholipid anion may include phosphatidylcholine, phosphatidylglycerol, phosphatidylserine, phosphatidylinositol, phosphatidylethanolamine, phosphocholine, or mixtures thereof. The hydrophobic anion may also exclude any anion described or any group of anions described. The hydrophobic anion may attach to a specific side chain on the protein or it may attach to multiple side chains on the physiologically active substance. The hydrophobic anion may have a log P greater than 1. The log P is the water-octanol partition coefficient and may be defined as the logarithm of the concentration of the protein salt in octanol to the concentration of the protein salt in water. A log P greater than 10 may result in a concentration in octanol that is 10 times greater than that in water. The water-octanol partition coefficient may be useful in comparing different molecules for their ability to partition into a hydrophobic phase, when the molecules themselves may be amphipathic.

The composition may include an inverted micelle. A micelle may be a molecule that has a hydrophilic head and a hydrophobic tail. The micelle may be referred to as inverted because the hydrophilic head faces inward and the hydrophobic tail faces outward. Inverted micelles may include phospholipids, DPPG, POPE, deoxycholic acid, sodium deoxycholate, sodium glycocholate, taurocholic acid sodium salt, N-dodecyl B-D-maltoside, tridecyl B-D-maltoside, SDS, DSS, DPC, and anions thereof. Inverted micelles may also be permeation enhancers.

The composition may include a biodegradable polymer. The biodegradable polymer may form a particle comprising the physiologically active substance. The biodegradable polymer may include PLGA or caprolactones. The PLGA may encompass the physiologically active substance, providing additional resistance against degradation in gastric acid. The biodegradable polymer may be insoluble in water. The biodegradable polymer may have carboxyl end groups, which ion pair with the physiologically active substance, making the physiologically active substance more likely to stay in the carrier fluid. The biodegradable polymer may act as a mucoadhesive substance and interact with the lining of the stomach.

The composition may include a pH modifier, e.g., a compound that increases the pH of the stomach. Increasing the pH of the stomach may counter the gastric acid and may delay the degradation of the physiologically active substance in the stomach. As examples, the composition may include sodium bicarbonate, which may raise the pH and decrease the activity of pepsin in the stomach. Other examples of gastric acid modulators include H₂ receptor blockers, proton pump inhibitors, prostaglandin E1-like compounds, and antacids, and salts thereof. Antacids may include sodium bicarbonate, potassium bicarbonate, calcium carbonate, calcium bicarbonate, aluminum bicarbonate, aluminum hydroxide, magnesium bicarbonate, magnesium hydroxide, magnesium trisilicate, and combinations thereof. Other gastric acid modulators are described in US Patent Publication No. 2017/0189363 A1, the contents of which are incorporated herein by reference for all purposes.

The composition may include an ionic or nonionic surfactant. Examples of ionic surfactants include sulfates, sulfonates, phosphates, carboxylates, ammonium lauryl sulfate, sodium lauryl sulfate, sodium laureth sulfate, sodium myreth sulfate, docusate (dioctyl sodium sulfosuccinate), perfluorooctanesulfonate (PFOS), perfluorobutanesulfonate, alkyl-aryl ether phosphates, and alkyl ether phosphates. Examples of nonionic surfactants include Triton X-100, Poloxamers, glycerol monostearate, glycerol monolaurate, sorbitan monolaurate, sorbitan monostearate, sorbitan tristearate, Tween 20, Tween 40, Tween 60, and Tween 80. The surfactant may help protect the oil phase from the acidic water phase.

The composition may include a peptidase inhibitor. Peptidase inhibitors may include ethylenediaminetetraacetic acid (EDTA) and soybean trypsin inhibitor (SBTI). Peptidase inhibitors may include any of the families of inhibitors, including Inhibitor I3A, Inhibitor I3B, Inhibitor 14, Inhibitor 19, Inhibitor 110, Inhibitor 124, Inhibitor 129, Inhibitor 134, Inhibitor 136, Inhibitor 142, Inhibitor 148, Inhibitor 153, Inhibitor 167, Inhibitor 168, and Inhibitor 178. Peptidase inhibitors are described in Rawlings et al., “Evolutionary families of peptidase inhibitors,” Biochem. J., 378(3) 705-716 (2004), the contents of which are incorporated by reference for all purposes. The composition may also exclude a peptidase inhibitor or include a peptidase inhibitor in lower concentrations than used in conventional oral delivery formulations.

The composition may not include an oil. The composition may exclude any compound or group of compounds described herein.

Several compounds may have properties of different compounds. For example, cyclodextrin may be a mucoadhesive substance and may be a stabilizer against acid and enzyme-catalyzed degradation. In some instances, the composition may include two, three, four, or five different compounds as the physiologically active substance, the carrier compound, the mucoadhesive compound, and the permeation enhancer. In some instances, the composition may include a single compound that acts as both a mucoadhesive and a permeation enhancer.

### III. STRUCTURE

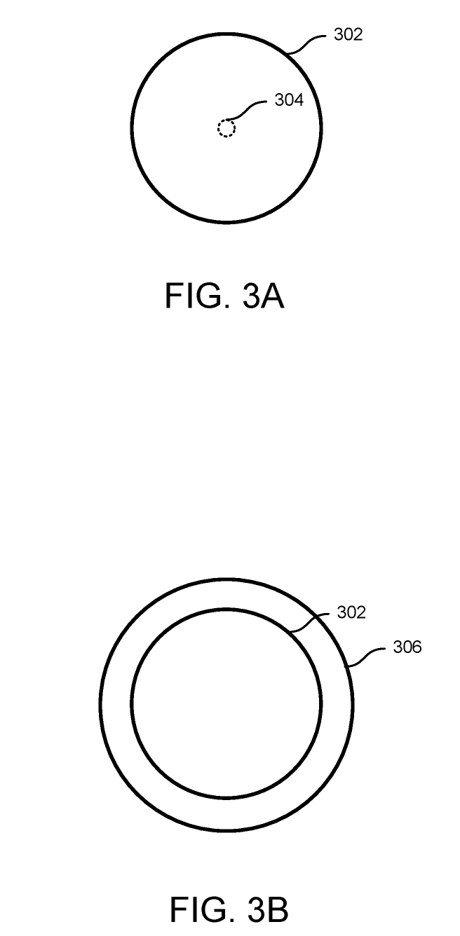

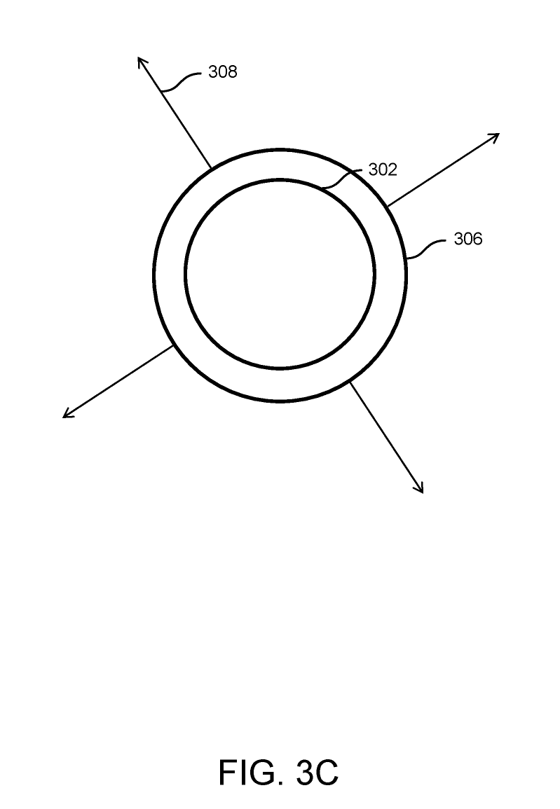

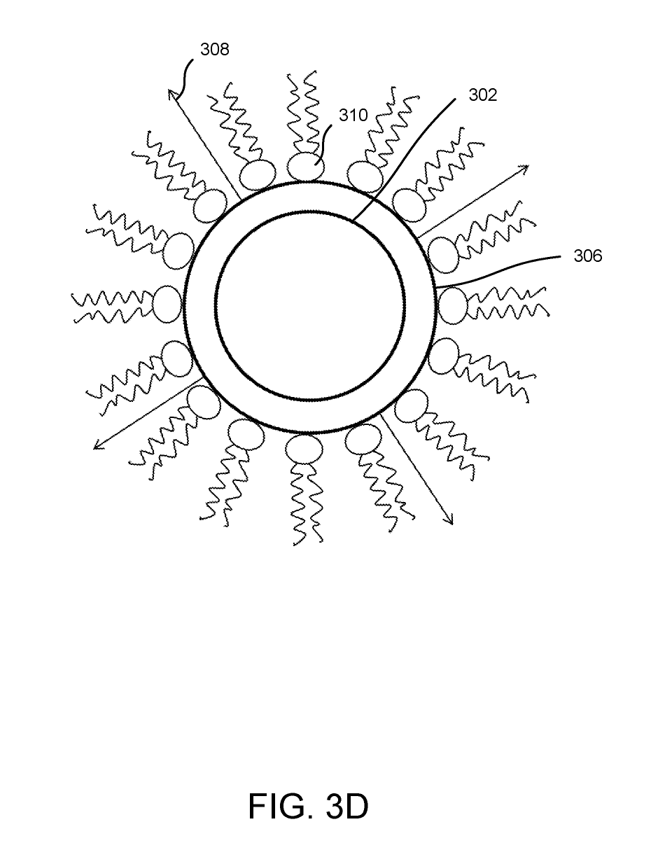

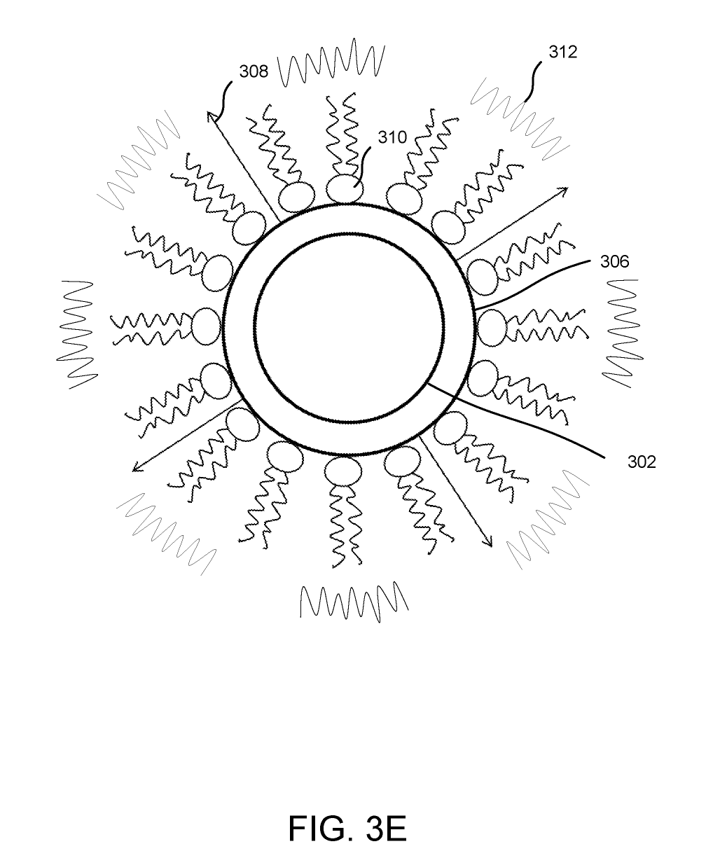

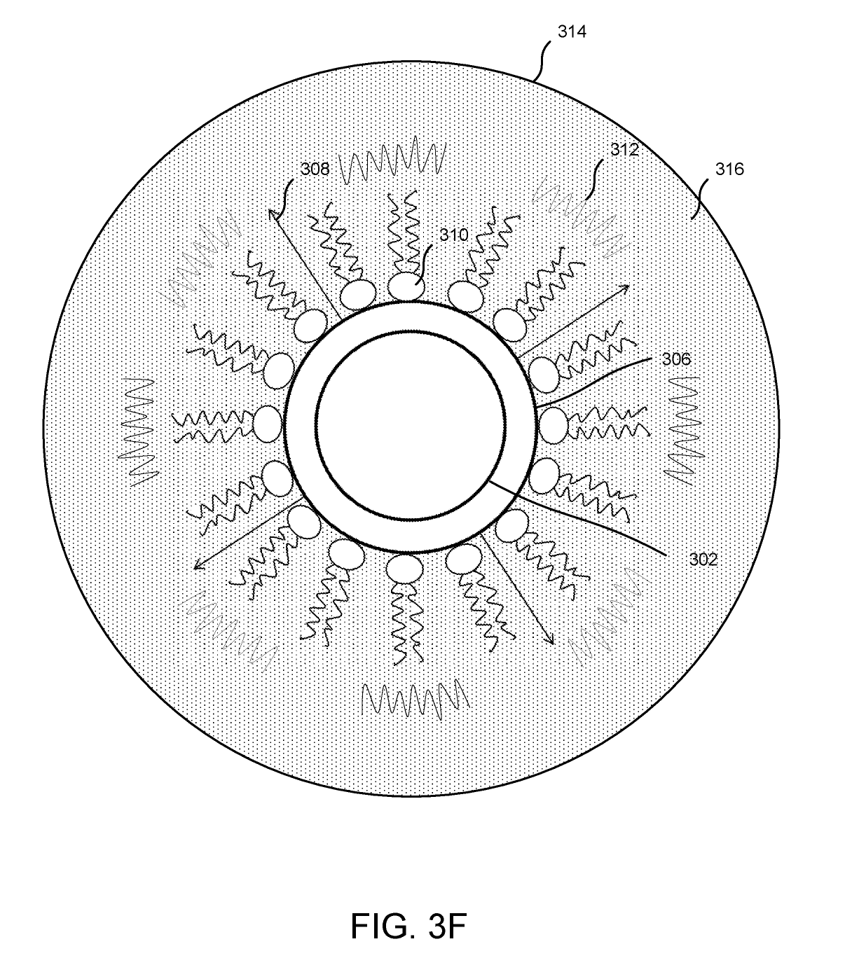

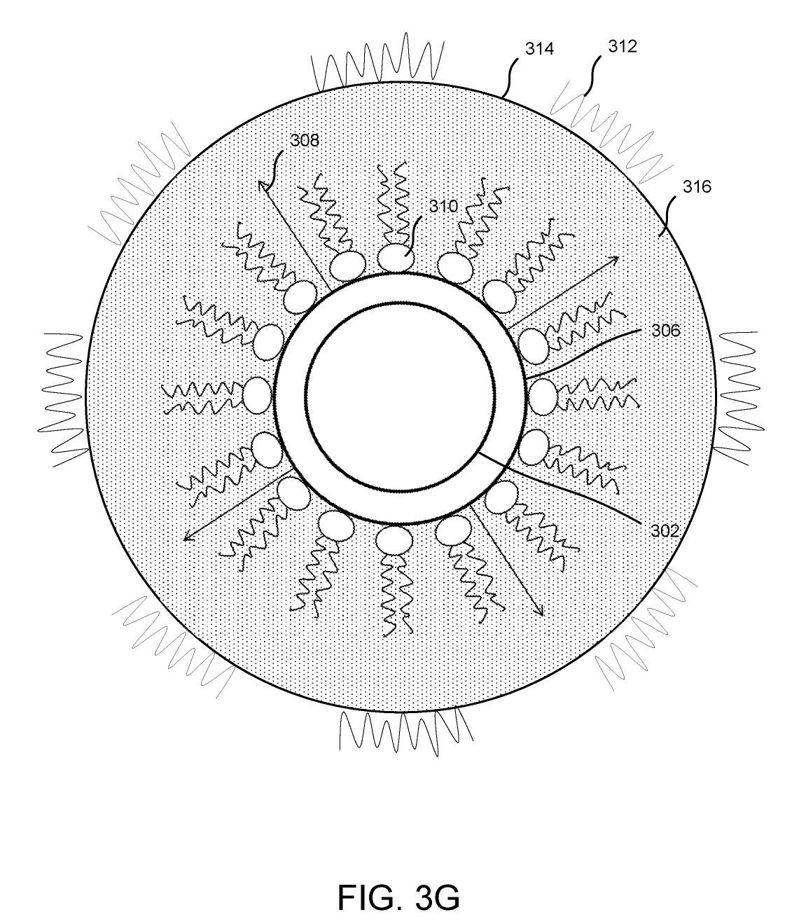

Embodiments may include a structure of a drug formulation for oral delivery, as shown in FIG. 3A-3F, which are not to scale. As in FIG. 3A, the drug formulation may include a physiologically active substance 302. Physiologically active substance 302 may be any physiologically active substance described herein. Physiologically active substance 302 may include a center of mass of the drug formulation 304.

The drug formulation may also include a material that includes at least one of a mucoadhesive compound, a permeation enhancer, an inverted micelle, or an inclusion compound in which the physiologically active substance forms an inclusion complex. The material may be in contact with the physiologically active substance. A portion of the material may be disposed farther from the center of mass than any portion of the physiologically active substance.

The material may include one, two, three, or four of the mucoadhesive compound, the permeation enhancer, the inverted micelle, or the inclusion compound. The material may also include at least one of a peptidase inhibitor, a pH modifier, or a surfactant.

As shown in FIG. 3B, the material may include inclusion compound 306 in which physiologically active substance 302 forms an inclusion complex. Inclusion compound 306 may include any compound described herein.

As shown in FIG. 3C, the material may include permeation enhancer 308. A portion of permeation enhancer 308 may be farther from the center of mass than any portion of inclusion compound 306. Permeation enhancer 308 may include any compound described herein.

As shown in FIG. 3D, the material may include inverted micelle 310. A portion of inverted micelle 310 may be farther from the center of mass than any portion of inclusion compound 306. Inverted micelle 310 may be any inverted micelle described herein.

As shown in FIG. 3E, the material may include mucoadhesive compound 312. A portion of mucoadhesive 312 may be farther from the center of mass than any portion of inclusion compound 306. Mucoadhesive compound may 312 be any mucoadhesive compound described herein.

Inclusion compound 306 may contact physiologically active substance 302. Inverted micelle 306 may contact inclusion compound 306. Permeation enhancer 308 may contact inclusion compound 306. Mucoadhesive 312 may contact at least one of inverted micelle 310 or permeation enhancer 308.

Not all compounds may be present. A portion of the mucoadhesive compound, if present, may be farther from the center of mass than any portion of the physiologically active substance, the permeation enhancer, the inverted micelle, or the inclusion compound. The inclusion compound, if present, may contact the physiologically active substance. The inverted micelle, if present, may contact the inclusion compound or the physiologically active substance. The permeation enhancer, if present, may contact the inclusion compound or the physiologically active substance. The compounds present may contact a compound nearer the center of mass. For example, a mucoadhesive compound may contact at least one of the permeation enhancer, the inverted micelle, the inclusion compound, or physiologically active substance.

As shown in FIG. 3F, the drug formulation may further include a capsule 314. Capsule 314 may encapsulate physiologically active substance 302, the material, and carrier compound 316. Carrier compound 316 may be any carrier compound described herein. FIG. 3F may be one embodiment of FIG. 2A. Capsule 314 may also encapsulate a peptidase inhibitor, a pH modifier, or a surfactant.

As shown in FIG. 3G, in some embodiments, mucoadhesive compound 312 may contact capsule 314 on a side of the capsule farther away from the center of mass of the drug formulation. Inside the capsule, the material may include at least one of permeation enhancer 308, inverted micelle 310, or inclusion compound 306. Capsule 314 may encapsulate the physiologically active substance and the material. Capsule may also encapsulate carrier compound 316. An additional mucoadhesive compound may be present inside capsule 314 and may be configured as in FIGS. 3E and 3F. FIG. 3G may be one embodiment of FIG. 2A.

The various layers from the physiologically active substance may serve as protective layers to keep the physiologically active substance from degrading in stomach acid.

### IV. METHODS OF MANUFACTURING

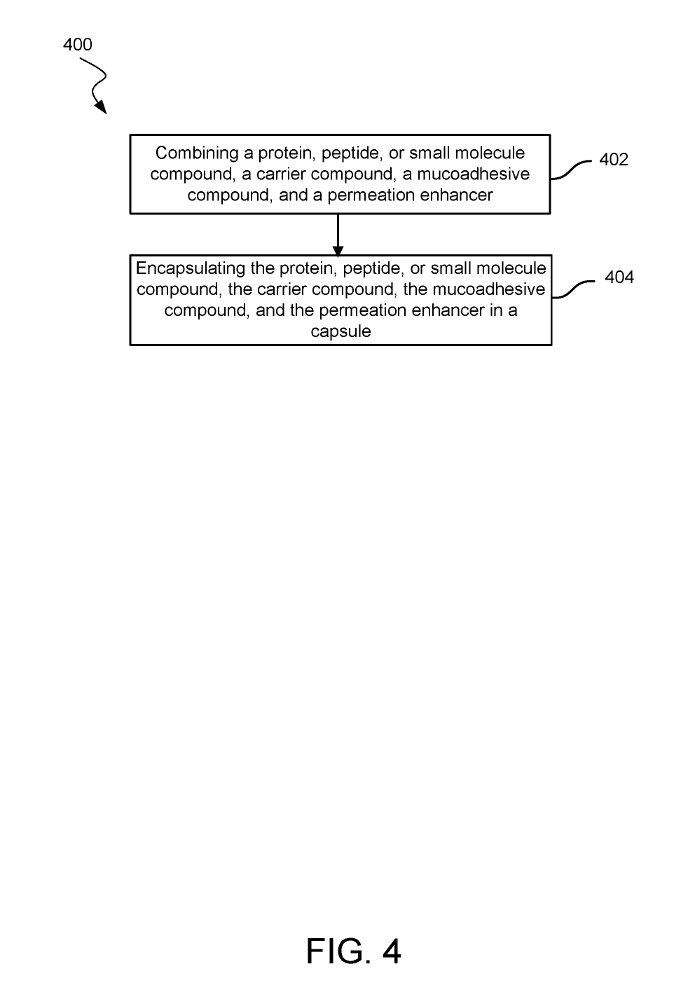

FIG. 4 shows a method 400 of manufacturing a drug for the oral delivery of a physiologically active substance. Method 400 may include combining a physiologically active substance, a carrier compound, a mucoadhesive compound, and a permeation enhancer (block 402). The physiologically active substance, the carrier compound, the mucoadhesive compound, and the permeation enhancer may be any compound described herein and may be combined in any of the amounts described herein. Method 400 may further include combining a peptidase inhibitor, a pH modifier, or a surfactant with the physiologically active substance. The peptidase inhibitor, pH modifier, and surfactant may be any disclosed herein. Any compound described herein may be excluded from being combined with the physiologically active substance.

An inclusion complex of the physiologically active substance may first be formed before block 402. Cyclodextrin or other cyclical compound may be mixed with the physiologically active substance in an aqueous solution. The inclusion complex may form a precipitate, which is the inclusion complex. The physiologically active substance may be in an inclusion complex when combined with other compounds.

The compounds may be combined and then agitated in some embodiments or not agitated in other embodiments. The compounds may be agitated by sonicating the mixture. The mixture may be sonicated at room temperature. The physiologically active substance, the carrier compound, the mucoadhesive may be sonicated together first before addition of the permeation enhancer. The mixture with the permeation enhancer may be briefly swirled or vortexed to mix. In some embodiments, method 400 may include coating the physiologically active substance with the carrier compound.

Method 400 may further include encapsulating the physiologically active substance, the carrier compound, the mucoadhesive compound, and the permeation enhancer in a capsule. The capsule may be configured to dissolve in gastric acid to release the physiologically active substance, the carrier compound, and the mucoadhesive compound. The capsule may be any capsule described herein. The capsule may include an enteric coating. In embodiments, the capsule may exclude an enteric coating, and the capsule and/or the composition in the capsule may exclude a peptidase inhibitor.

### V. METHODS OF TREATMENT

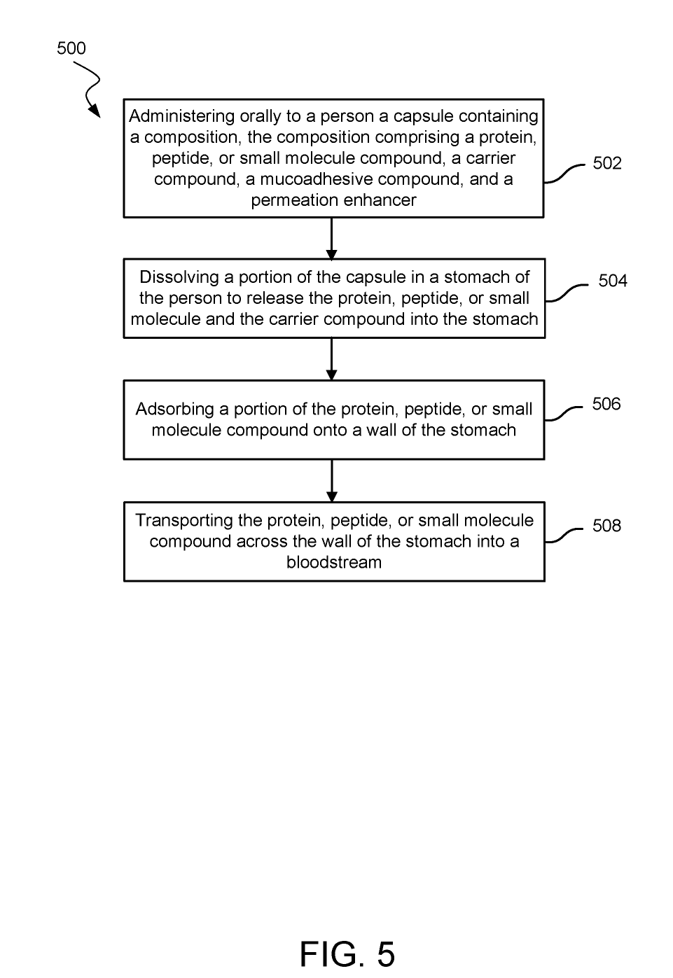

FIG. 5 shows a method 500 of treatment. The treatment may include a treatment for a disorder affecting metabolic pathways. The disorder may include diabetes, a growth deficiency, HIV, AIDS, a bone disorder, or osteoporosis.

Method 500 may include orally administering to a person a capsule containing a composition (block 502). The composition may include a physiologically active substance, a carrier compound, a mucoadhesive compound, and a permeation enhancer. The composition may also include at least one of a peptidase inhibitor, a pH modifier, or a surfactant. The composition may be any composition described herein.

Method 500 may also include dissolving a portion of the capsule in a stomach of the person to release the physiologically active substance and the carrier compound into the stomach (block 504).

Method 500 may further include adsorbing a portion of the physiologically active substance onto a wall of the stomach (block 506). Before the portion of the physiologically active substance adsorbs onto the wall of the stomach, the portion of the physiologically active substance may remain in the carrier compound. Because the carrier may be immiscible in the gastric acid, the physiologically active substance may not degrade before being adsorbed onto the stomach wall.

In addition, method 500 may include transporting the physiologically active substance across the wall of the stomach into a bloodstream (block 508). Transporting the physiologically active substance across the wall of the stomach may be about 3 to 4 hours after administering orally the capsule.

### VI. EXAMPLES

Human insulin was used in the examples unless otherwise noted.

### A. EXAMPLE 1

Three samples, all with 3 mg of insulin-PEG conjugate (with a 5 kDa PEG) and 1 mL fish oil, were prepared.

Sample 1: 3 mg of insulin-PEG conjugate, 1 mL fish oil, 50 mg β-cyclodextrin, and 3 mg dodecylphosphocholine (DPC).

Sample 2: 3 mg of insulin-PEG conjugate, 1 mL fish oil, 0.7 mg pamoic acid.

Sample 3: 3 mg of insulin-PEG conjugate, 1 mL fish oil, 3 mg DPC.

Samples 1-3 were sonicated until they appeared cloudy but homogenous. The samples were then added to 5 mL of simulated gastric fluid that did not include pepsin. The mixtures were inverted several times to mix.

The samples were run through High Performance Liquid Chromatography (HPLC) to determine if the insulin-PEG remained in the oil phase. HPLC showed that the insulin-PEG left the oil phase and entered the gastric fluid phase within 15 minutes for samples 1-3. None of the samples in this example were observed to be suitable for oral delivery of insulin because the insulin-PEG conjugate did not remain in the oil phase long enough.

### B. Example 2

Pamoate salts of insulin-PEG (5 kDa) were tested to see if the insulin-PEG pamoate salt would remain in the oil phase longer. The insulin-PEG pamoate salt was prepared by mixing insulin-PEG with sodium pamoate at a pH above 7. The pH was then reduced to 4. The precipitate was then collected and dried by lyophilization. The insulin-PEG pamoate salt was included in samples 4 and 6. In sample 5, sodium pamoate was added to insulin-PEG without forming the insulin-PEG pamoate salt.

Sample 4: 3 mg of insulin-PEG pamoate salt, 1 mL fish oil, 50 mg β-cyclodextrin, 3 mg DPC.

Sample 5: 3 mg insulin-PEG, 0.5 mg sodium pamoate, 1 mL fish oil, 50 mg β-cyclodextrin, 3 mg DPC.

Sample 6: 3 mg of insulin-PEG pamoate salt, 1 mL fish oil, 3 mg DPC.

Samples 4-6 were sonicated until cloudy and homogenous. The samples were then added to 5 mL of simulated gastric fluid (without pepsin). The mixtures were inverted several times to mix.

The samples were run through High Performance Liquid Chromatography (HPLC) to determine if the insulin-PEG remained in the oil phase. With sample 5, the insulin-PEG left the oil phase and was found in the gastric fluid phase within 15 minutes. With sample 6, the insulin-PEG stayed in the oil phase for at least 1.5 hours and then was found in the gastric fluid phase. With sample 4, only a small amount of insulin-PEG was found in the gastric fluid phase even at 5 hours, either the insulin stayed in the oil phase or precipitated.

The samples in this example appeared to show that the insulin-PEG pamoate salt would stay in the oil phase longer than when sodium pamoate was added to insulin-PEG. Sample 4 showed the most suitable results for oral delivery of insulin, which may be a result of the β-cyclodextrin.

### C. Example 3

Inclusion complexes of insulin-PEG (5 kDa) and α-cyclodextrin were tested. Insulin-PEG and 10 molar excess α-cyclodextrin were mixed in an aqueous solution and kept overnight at 4° C. to form a precipitate. The resulting precipitate was lyophilized and used in sample 7.

Sample 7: 6.2 mg insulin-PEG and α-cyclodextrin inclusion complex, 1 mL fish oil, 3 mg DPC.

Sample 8: 3.1 mg insulin-PEG, 1 mL fish oil, 3 mg DPC.

In forming samples 7 and 8, the insulin-PEG (or insulin-PEG inclusion complex) and the fish oil were sonicated for 30 minutes. The DPC was then added to the sonicated mixture and the mixture was briefly swirled or vortexed to mix. The resulting mixtures were kept at room temperature for 1 hour. The samples were then added to 5 mL of simulated gastric fluid (without pepsin). The mixtures were inverted several times to mix.

HPLC determined that with sample 7, 35% of the inclusion complex remained in the oil phase after 3 hours. In the same time, sample 8 had all of the insulin-PEG partition into the water phase. This example showed that an insulin-PEG inclusion complex stayed in the oil phase longer than the insulin-PEG conjugate that was not part of an inclusion complex.

### D. Example 4

An inclusion complex was tested in an aqueous solution of pepsin.

Sample 9: 2 mg insulin-PEG (5 kDa) and α-cyclodextrin inclusion complex.

Sample 9 was added to an aqueous solution of 1 mg pepsin in 1 mL simulated gastric fluid. The insulin-PEG was completely digested. This example shows that the inclusion complex was not enough to protect the insulin from degradation at the tested concentration.

### E. Example 5

Sample 7 of Example 3 was added to simulated gastric fluid that contained 1 mg/ml pepsin. HPLC showed that about 12% of insulin-PEG was present in the oil phase after 3 hours. This example suggests that the inclusion complex protects degradation of the insulin-PEG when the insulin-PEG remains in the oil phase.

### F. Example 6

Inclusion complexes with α-cyclodextrin, β-cyclodextrin, and γ-cyclodextrin were tested. α-cyclodextrin has the smallest doughnut hole formed by the ring, while γ-cyclodextrin has the largest. Insulin-PEG (5 kDa) and 10 molar excess of either α-cyclodextrin, β-cyclodextrin, or γ-cyclodextrin were mixed in an aqueous solution and kept overnight at 4° C. to form a precipitate. The resulting precipitates were lyophilized. Partitioning studies were performed in simulated gastric fluid as in Example 1. After 3 hours, 35% of the inclusion complex with α-cyclodextrin, 7% of the inclusion complex with β-cyclodextrin, and 10% of the inclusion complex with γ-cyclodextrin remained in the oil phase. This example showed that the α-cyclodextrin inclusion complex had the best performance for the insulin-PEG with a 5 kDa PEG. The α-cyclodextrin may have a more suitable size doughnut hole for the insulin-PEG than the other cyclodextrins.

### G. Example 7

An insulin-PEG with 2 kDa PEG was tested both in an inclusion complex and not including an inclusion complex. The insulin-PEG and 10 molar excess α-cyclodextrin were mixed in an aqueous solution and kept overnight at 4° C. to form a precipitate. The resulting precipitate was lyophilized and used in sample 10.

Sample 10: 6.2 mg insulin-PEG and α-cyclodextrin inclusion complex, 1 mL fish oil, 3 mg DPC.

Sample 11: 3.1 mg insulin-PEG, 1 mL fish oil, 3 mg DPC.

In forming samples 10 and 11, the insulin-PEG (or insulin-PEG inclusion complex) and the fish oil were sonicated for 30 minutes. The DPC was then added to the sonicated mixture and the mixture was briefly swirled or vortexed to mix. The resulting mixtures were kept at room temperature for 1 hour. The samples were then added to 5 mL of simulated gastric fluid (without pepsin). The mixtures were inverted several times to mix.

HPLC determined that with sample 10, 20% of the inclusion complex remained in the oil phase after 3 hours. In the same time, sample 11 had 6% of the insulin-PEG remaining in the oil phase. This example showed that an insulin-PEG inclusion complex stayed in the oil phase longer than the insulin-PEG conjugate that is not part of an inclusion complex. Additionally, sample 11 had a larger amount of the insulin-PEG remain in the oil phase than sample 8 of Example 3. The results of Example 7 show that insulin-PEG with 2 kDa PEG stayed in the oil better than insulin-PEG with 5 kDa PEG.

### H. Example 8

In order to estimate the membrane permeability of various formulations, a Caco-2 permeability study was performed. The compounds tested were insulin-PEG (5 kDa), insulin-PEG (5 kDa) inclusion complex, insulin-PEG (5 kDa) inclusion complex and DPC, insulin-PEG (2 kDa), and the insulin-PEG (2 kDa) inclusion complex. All compounds were dissolved in media at a concentration of 1 mg insulin/mL media and added to a Caco-2 monolayer. The insulin-PEG (2 kDa) had 1% of the insulin-PEG permeate through the cell layer after 3 hours. The insulin-PEG (2 kDa) inclusion complex had 5% of the insulin-PEG permeate through the cell layer after 3 hours. None of the other compounds showed any permeation through the cell layer after 3 hours. This study showed that insulin-PEG could permeate through a Caco-2 intestinal cell layer. Based on these results, insulin-PEG may be expected to be able to permeate through the stomach wall.

### I. Example 9

An in vivo study was performed with different insulin-PEG samples and a vehicle group with fish oil alone.

Sample 12: 3.1 mg insulin-PEG (5 kDa), 1 mL fish oil, 3 mg DPC.

Sample 13: 6.2 mg insulin-PEG (5 kDa) and α-cyclodextrin inclusion complex, 1 mL fish oil, 3 mg. DPC.

Sample 14: 2.1 mg insulin-PEG (2 kDa), 1 mL fish oil, 3 mg DPC.

Sample 15: 4.87 mg insulin-PEG (2 kDa) and α-cyclodextrin inclusion complex, 1 mL fish oil, 3 mg DPC.

In forming samples 12-15, the insulin-PEG (or insulin-PEG inclusion complex) and the fish oil were sonicated for 30 minutes. The DPC was then added to the sonicated mixture and the mixture was briefly swirled or vortexed to mix. The resulting mixtures were kept at room temperature for 1 hour. The samples were administered via an oral gavage to rats at 150 IU/kg (for 5 kDa PEG) and 75 IU/kg (for 2 kDa PEG). At specified intervals, blood was collected from the jugular vein and analyzed for glucose values.

A significant glucose reduction was observed in some rats dosed with insulin-PEG formulations. One rat in each group dosed with the 5 kDa insulin-PEG (samples 12 and 13) had a significant glucose reduction to ≤20 mg/dL within 30 minutes. The glucose levels for these rats gradually increased over the next few hours and were back at baseline (˜50 mg/dL) by approximately three hours. The remaining rats in these groups had a glucose response similar to the control group. Two of the rats dosed with sample 14 (2 kDa PEG) had glucose reductions to ≤20 mg/dL within 30 minutes, the glucose remained low for three hours at which point these two rats had to be given dextrose because their glucose levels were too low. The remaining rats in that group had a glucose response similar to the control group. Two of the rats dosed with sample 15 (2 kDa PEG) had glucose reductions to ≤20 mg/dL within 30 minutes, one of these rats had glucose levels close to baseline after 2 hours, the other rat's glucose level remained low for three hours at which point they had to be given dextrose because their glucose levels were too low. The remaining rats in this group had a glucose response similar to the control group. The rats that had a significant glucose response also had detectable levels of Insulin-PEG in the blood serum as detected by ELISA. Insulin-PEG with 2 kDa PEG appeared to be better absorbed than 5 kDa PEG in certain rats, as the amount of serum insulin-PEG was greater in those rats. This resulted in a more reproducible and prolonged glucose reduction with 2 kDa insulin-PEG, which is consistent with the findings from Example 8.

### J. Example 10

An in vivo study was performed to compare an oral insulin-PEG sample to subcutaneous injections of insulin-PEG and insulin.

Sample 16: 2.1 mg insulin-PEG (2 kDa), 1 mL fish oil, 3 mg DPC.

Sample 17: 0.015 mg/kg insulin-PEG (2 kDa)

Sample 18: 0.011 mg/kg insulin

In forming sample 16, the insulin-PEG and the fish oil were sonicated for 30 minutes. The DPC was then added to the sonicated mixture and the mixture was briefly swirled or vortexed to mix. The resulting mixtures were kept at room temperature for 1 hour. Sample 16 was administered via an oral gavage to rats at 40 and 60 IU/kg. Samples 17 and 18 were administered subcutaneously at 0.3 IU/kg. At specified intervals, blood was collected from the jugular vein and analyzed for glucose values.

All rats dosed subcutaneously with samples 17 and 18 had a reduction in blood glucose to ≤20 mg/dL within 30 minutes. Sample 17 (insulin-PEG) had a gradual increase in glucose and was back to baseline (˜60 mg/dL) by about 4 hours. Sample 18 (insulin) had a gradual increase in glucose and was back to baseline by about 3 hours. One rat dosed orally with 40 IU/kg of sample 16 had a reduction in glucose to 40 mg/dL at the 30 minutes timepoint and one rat had a reduction to 40 mg/dL at the 6 hour timepoint, the glucose levels of these rats were back to baseline at the next timepoint. The remaining rats did not have a significant glucose reduction. Two rats dosed orally with 60 IU/kg of sample 16 had a reduction in glucose to 40 mg/dL at the 2 hour timepoint, which returned to baseline by 3 hours, and one rat had a reduction at the eight hour timepoint to 30 mg/dL. The remaining rats did not have a significant glucose reduction. Although there was some glucose lowering seen from the oral formulations at 40 and 60 IU/kg, the response was not as significant as that seen in Example 9 where the administered dose was higher.

### K. Example 11

An in vivo study was performed to compare an oral insulin-PEG sample to a subcutaneous injection of insulin-PEG. This study was similar to Example 10, except that the oral formulation was dosed at 75 IU/kg.

Sample 19: 0.011 mg/kg insulin

Sample 20: 2.1 mg insulin-PEG (2 kDa), 1 mL fish oil, 3 mg DPC.

In forming sample 20, the insulin-PEG and the fish oil were sonicated for 30 minutes. The DPC was then added to the sonicated mixture and the mixture was briefly swirled or vortexed to mix. The resulting mixtures were kept at room temperature for 1 hour. Sample 20 was administered via an oral gavage to rats at 75 IU/kg. Sample 19 was administered subcutaneously at 0.3 IU/kg. At specified intervals, blood was collected from the jugular vein and analyzed for glucose values.

All rats dosed subcutaneously with sample 19 had a reduction in blood glucose to 20 mg/dL within 30 minutes, the levels gradually increased and were back to baseline (˜60 mg/dL) by about 3 hours. Three of five rats dosed orally with sample 20 had a reduction in blood glucose of between 20 and 40 mg/dL (at least a 30% reduction) at 30 minutes, the levels gradually increased and were back to baseline by about 3 hours. The remaining two rats did not have a significant glucose reduction. These results were similar to sample 14.

### L. Example 12

Seventeen samples were prepared with different protein/peptide Active Pharmaceutical Ingredients (APIs) (either insulin-PEG conjugate (with a 2 kDa PEG) or GLP-1 (with a 5 kDa PEG)), permeation enhancers, mucoadhesive compounds, and carrier compounds. Some compounds can function as both permeation enhancers and mucoadhesive compounds.

Sample 21: 2 mg of insulin-PEG conjugate, 1 mL fish oil

Sample 22: 2 mg of insulin-PEG conjugate, 3 mg DPC, 1 mL fish oil.

Sample 23: 2 mg of insulin-PEG conjugate, 3 mg 1,2-dipalmitoyl-sn-glycerol-3-phosphoglycerol (DPPG), 1 mL fish oil.

Sample 24: 2 mg of insulin-PEG conjugate, 3 mg 1-palmitoyl-2-oleoyl-sn-glycero-3-phosphoethanolamine (POPE), 1 mL fish oil.

Sample 25: 2 mg of insulin-PEG conjugate, 3 mg deoxycholic acid, 1 mL fish oil.

Sample 26: 2 mg of insulin-PEG conjugate, 3 mg sodium deoxycholate, 1 mL fish oil.

Sample 27: 2 mg of insulin-PEG conjugate, 3 mg sodium glycholate, 1 mL fish oil.

Sample 28: 2 mg of insulin-PEG conjugate, 3 mg taurocholic acid sodium salt, 1 mL fish oil.

Sample 29: 2 mg of insulin-PEG conjugate, 3 mg N-dodecyl B-D-maltisidase, 1 mL fish oil.

Sample 30: 2 mg of insulin-PEG conjugate, 3 mg tridecyl B-D-maltisidase, 1 mL fish oil.

Sample 31: 2 mg of insulin-PEG conjugate, 3 mg sodium dodecyl sulfate (SDS), 1 mL fish oil.

Sample 32: 2 mg of GLP-1-PEG conjugate, 3 mg DPC, 1 mL fish oil.

Sample 33: 2 mg of insulin-PEG conjugate, 3 mg DPC, 1 mL krill oil.

Sample 34: 2 mg of insulin-PEG conjugate, 3 mg DPC, 3 mg SDS, 1 mL fish oil.

Sample 35: 2 mg of insulin-PEG conjugate, 3 mg DPC, 3 mg sodium docusate (DSS), 1 mL fish oil.

Sample 36: 2 mg of insulin-PEG conjugate, 3 mg DPC, 50 mg Hepakis 2,6-B-O-methyl-B-cyclodextrin, 1 mL fish oil.

Sample 37: 2 mg of insulin-PEG conjugate, 3 mg DPC, 50 mg Polylactide-co-glycolide (PLGA), 1 mL fish oil.

The peptide and oil for samples 21-37 were sonicated until they appeared cloudy but homogenous. Then the remaining ingredients were added to each sample. The samples were sonicated again until they appeared cloudy but homogenous. The samples were then added to 5 mL of simulated gastric fluid that did not include pepsin. The mixtures were inverted several times to mix.

Samples of the gastric fluid phase were analyzed by High Performance Liquid Chromatography (HPLC) to determine if the insulin-PEG remained protected in the oil phase and how much went into the water phase. HPLC showed that in most of the formulations the insulin-PEG left the oil phase and entered the gastric fluid phase within 15 minutes. Some results showed greater than 100% API as a result of experimental and/or other error. In samples 23, 24, and 31 the insulin-PEG appeared to leave the oil phase more slowly, suggesting that DPPG, POPE, and DSS help to keep the insulin in the oil.

TABLE 1

| | % API in | | |
|---|---|---|---|
| | water phase | % API in water | % API in water |
| Sample # | 0.25 hr | phase 1 hr | phase 3 hr |
| 21 | 118.0 | 100.0 | 75.2 |
| 22 | 88.4 | 70.3 | 48.2 |
| 23 | 50.4 | 60.0 | 53.2 |
| 24 | 16.4 | 34.0 | 55.1 |
| 25 | 82.4 | 88.1 | 75.0 |
| 26 | not determined | 70.6 | 65.8 |
| 27 | 134.1 | 85.0 | 82.5 |
| 28 | 91.1 | 97.0 | 82.6 |
| 29 | 78.6 | 97.6 | 83.0 |
| 30 | 106.8 | 96.9 | 84.6 |
| 31 | 59.2 | 87.4 | 70.9 |
| 32 | not determined | not determined | not determined |
| 33 | not determined | not determined | not determined |
| 34 | 75.8 | 68.0 | 79.7 |
| 35 | 73.8 | 40.7 | 43.1 |
| 36 | 87.0 | 67.6 | 65.5 |
| 37 | 44.4 | 41.1 | 50.1 |

### M. Example 13

Six samples were prepared with insulin-PEG conjugate (with 2 kDa PEG), with different permeation enhancers, mucoadhesive compounds, and carrier compounds. Some compounds can function as both permeation enhancers and mucoadhesive compounds.

Sample 38: 3 mg of insulin-PEG conjugate, 6 mg tridecyl-B-maltoside, 1 mL fish oil

Sample 39: 3 mg of insulin-PEG conjugate, 4 mg 1-palmitoyl-2-oleoyl-sn-glycero-3-phosphoethanolamine (POPE), 1 mL fish oil.

Sample 40: 3 mg of insulin-PEG conjugate, 4 mg DPC, 1 mL olive oil.

Sample 41: 3 mg of insulin-PEG conjugate, 4 mg DPC, 50 mg PLGA, 1 mL olive oil.

Sample 42: 3 mg of insulin-PEG conjugate, 4 mg POPE, 1 mL olive oil.

Sample 43: 3 mg of insulin-PEG conjugate, 4 mg POPE, 4 mg DPC, 1 mL olive oil.

The peptide and oil for samples 38-43 were sonicated until they appeared cloudy but homogenous. Then the remaining ingredients were added to each sample. The samples were sonicated again until they appeared cloudy but homogenous. The samples were then added to 5 mL of simulated gastric fluid that did not include pepsin. The mixtures were inverted several times to mix.

The samples were run through High Performance Liquid Chromatography (HPLC) to determine if the insulin-PEG remained in the oil phase and how much went into the water phase. HPLC showed that in most of the formulations the insulin-PEG left the oil phase and entered the gastric fluid phase within 15 minutes. In samples 39 and 42 the insulin-PEG appeared to leave the oil phase more slowly, again suggesting that POPE helps to retain the insulin-PEG in the oil phase. However, POPE in the presence of DPC, which is important for insulin-PEG absorption, did not appear to contribute to keeping the insulin-PEG in the oil.

TABLE 2

| | % insulin-PEG | % insulin-PEG | % insulin-PEG |
|---|---|---|---|
| | in water phase | in water phase | in water phase |
| Sample # | 0.25 hr | 1 hr | 3 hr |
| 38 | 89.0 | 82.1 | 81.0 |
| 39 | 54.9 | 45.3 | 42.1 |
| 40 | 95.2 | 101.5 | 100.2 |
| 41 | 103.9 | 105.9 | 101.5 |
| 42 | 22.8 | 52.5 | 48.1 |
| 43 | 113.3 | 128.2 | 125.3 |

### N. Example 14

An in vivo study was performed to compare oral insulin-PEG samples to a subcutaneous injection of insulin. Four formulations were prepared for oral delivery, containing insulin-PEG conjugate (with 2 kDa PEG) with different inclusion complexes, permeation enhancers, mucoadhesive compounds, and carrier compounds as detailed in Table 3.

TABLE 3

| | Sample | | Sample | | Sample | | Sample | | Sample |
|---|---|---|---|---|---|---|---|---|---|
| | 44 | | 45 | | 46 | | 47 | | 48 |
| Olive Oil | 1 | ml | 1 | ml | 1 | ml | 1 | ml | |
| Insulin-PEG (2 kDa) | | | 2.1 | mg | | | 2.1 | mg | |
| Insulin-PEG (2 kDa)/ | 4.8 | mg | | | 4.8 | mg | | | |
| α-cyclodextrin | | | | | | | | | |
| POPE | 30 | mg | 30 | mg | 30 | mg | 30 | mg | |
| DPC | 3 | mg | 3 | mg | 3 | mg | 3 | mg | |
| PLGA | 50 | mg | 50 | mg | | | | | |

| | | | | | |
|---|---|---|---|---|---|
| Humulin-R SC dose | | | | | ~0.011 |
| (μg/kg) | | | | | |
| Treatment Dose | 75 | 75 | 75 | 75 | 0.3 |
| (Insulin equivalent | | | | | |
| IU/kg) | | | | | |

The peptide and oil for formulations 44-47 were sonicated until they appeared cloudy but homogenous. Then the remaining ingredients were added to each sample. The samples were sonicated again until they appeared cloudy but homogenous. The samples were stored overnight at room temperature. Normal SD Rats (5 rats/group) were fasted overnight before being dosed by oral gavage at insulin equivalent dosage of 75 IU/kg. For comparison, a fifth group (sample 48) was dosed with Humulin R (recombinant human insulin) subcutaneously at 0.3 IU/kg. Blood was collected at predose (−30 min) and post dose (10 min, 30 min, 1 h, 1.5 h, 2 h, 3 h, 4 h, 6 h, 8 h) for glucose measurements by a glucometer.

All rats dosed subcutaneously with sample 48 had a reduction in blood glucose to ≤20 mg/dL within 30 minutes. The glucose levels gradually increased and were back at baseline at 3 hours. Three of five rats given sample 44 had at least a 30% reduction in blood glucose, with the minimum occurring at 2 or 3 hours. At the four hour time point, the blood glucose was back to baseline in these rats, except for one rat whose blood glucose remained below baseline level through 6 hours. One rat given sample 47 had a significant glucose reduction, with the minimum at 2 hours. The blood glucose slowly returned to baseline levels, but not until 8 hours. Two rats given sample 45 had a glucose reduction to ≤20 mg/dL within 30 minutes, while one rat had a 30% reduction at 2 hours. One rat give sample 46 had a glucose reduction to ≤20 mg/dL within 30 minutes. These rats returned to baseline glucose levels about two hours after the minimum glucose levels were reached. It should be noted that at the ten minute blood draw two rats given sample 45 and one rat given sample 46 were observed to have oil around its mouth, coinciding with the rats that had glucose reductions. The oil around the mouth suggested that the dose may not be entirely delivered into the stomach of these rats, and these rats had a significant glucose reduction at 30 minutes.

### O. Example 15

Six samples were prepared with insulin-PEG conjugate (with 2 kDa PEG), with different mucoadhesive compounds or permeation enhancers.

Sample 49: 3.68 mg insulin-PEG (2 kDa) and α-cyclodextrin inclusion complex, 15 mg POPE, 2.6 mg DPC, 0.8 mL olive oil.

Sample 50: 3.68 mg insulin-PEG (2 kDa) and α-cyclodextrin inclusion complex, 15 mg POPE, 2.6 mg DPC, 10.6 mg Poloxamer 188, 0.8 mL olive oil.

Sample 51: 3.68 mg insulin-PEG (2 kDa) and α-cyclodextrin inclusion complex, 15 mg POPE, 2.6 mg DPC, 10 mg low molecular weight chitosan, 0.8 mL olive oil.

Sample 52: 3.68 mg insulin-PEG (2 kDa) and α-cyclodextrin inclusion complex, 15 mg POPE, 2.6 mg DPC, 10 mg low molecular weight chitosan, 25 mg DSS, 0.8 mL olive oil.

Sample 53: 3.68 mg insulin-PEG (2 kDa) and α-cyclodextrin inclusion complex, 15 mg POPE, 2.6 mg DPC, 10 mg carboxymethylcellulose, 25 mg DSS, 0.8 mL olive oil.

Sample 54: 3.68 mg insulin-PEG (2 kDa) and α-cyclodextrin inclusion complex, 15 mg POPE, 2.6 mg DPC, 40 mg PLGA, 0.8 mL olive oil.

The peptide and oil for samples 49-54 were sonicated until they appeared cloudy but homogenous. Then the remaining ingredients were added to each sample. The samples were sonicated again until they appeared cloudy but homogenous. The samples were then added to 5 mL of simulated gastric fluid that did not include pepsin. The mixtures were inverted several times to mix.

The samples were run through High Performance Liquid Chromatography (HPLC) to determine if the insulin-PEG remained in the oil phase and how much went into the water phase. In samples 52 and 53, which contained DSS, the insulin-PEG appeared to leave the oil phase more slowly, suggesting that DSS is helping to keep the insulin-PEG in the oil phase.

TABLE 4

| | % insulin-PEG in | % insulin-PEG in |
|---|---|---|
| Sample # | water phase 0.25 hr | water phase 1 hr |
| 49 | 37.2 | 71.1 |
| 50 | 34.0 | 56.1 |
| 51 | 27.7 | 45.8 |
| 52 | 16.6 | 16.0 |
| 53 | 5.2 | 6.1 |
| 54 | 16.6 | 47.9 |

### P. Example 16

Nine samples were prepared with insulin-PEG conjugate (with 2 kDa PEG), with different types of and amounts of permeation enhancers. The different permeation enhancers included DPC, DSS, and POPE.

Sample 55: 3.68 mg insulin-PEG (2 kDa) and α-cyclodextrin inclusion complex, 2.4 mg DPC, 0.8 mL olive oil.

Sample 56: 3.68 mg insulin-PEG (2 kDa) and α-cyclodextrin inclusion complex, 3 mg POPE, 2.6 mg DPC, 0.8 mL olive oil.

Sample 57: 3.68 mg insulin-PEG (2 kDa) and α-cyclodextrin inclusion complex, 15 mg POPE, 2.6 mg DPC, 0.8 mL olive oil.

Sample 58: 3.68 mg insulin-PEG (2 kDa) and α-cyclodextrin inclusion complex, 3 mg POPE, 3 mg DSS, 2.6 mg DPC, 0.8 mL olive oil.

Sample 59: 3.68 mg insulin-PEG (2 kDa) and α-cyclodextrin inclusion complex, 3 mg POPE, 30 mg DSS, 2.6 mg DPC, 0.8 mL olive oil.

Sample 60: 3.68 mg insulin-PEG (2 kDa) and α-cyclodextrin inclusion complex, 15 mg POPE, 3 mg DSS, 2.6 mg DPC, 0.8 mL olive oil.

Sample 61: 3.68 mg insulin-PEG (2 kDa) and α-cyclodextrin inclusion complex, 15 mg POPE, 30 mg DSS, 2.6 mg DPC, 0.8 mL olive oil.

Sample 62: 3.68 mg insulin-PEG (2 kDa) and α-cyclodextrin inclusion complex, 3 mg DSS, 2.6 mg DPC, 0.8 mL olive oil.

Sample 63: 3.68 mg insulin-PEG (2 kDa) and α-cyclodextrin inclusion complex, 30 mg DSS, 2.6 mg DPC, 0.8 mL olive oil.

The peptide and oil for samples 55-63 were sonicated until they appeared cloudy but homogenous. Then the remaining ingredients were added to each sample. The samples were sonicated again until they appeared cloudy but homogenous. The samples were then added to 5 mL of simulated gastric fluid that did not include pepsin. The mixtures were inverted several times to mix.

The samples were run through High Performance Liquid Chromatography (HPLC) to determine if the insulin-PEG remained in the oil phase and how much went into the water phase. In sample 58, which contained equivalent amounts of DSS and POPE, and samples 59 and 61, which contained more DSS than POPE, the insulin-PEG appeared to leave the oil phase more slowly. The amount of insulin-PEG that left the oil phase in samples 62 and 63, which had DSS but not POPE, was more than in samples 58, 59, and 61, indicating that DSS and POPE may act together to keep insulin-PEG in the oil phase.

TABLE 5

| | % insulin-PEG in | % insulin-PEG in |
|---|---|---|
| Sample # | water phase 0.5 hr | water phase 2.5 hr |
| 55 | 60.9 | 115.2 |
| 56 | 53.7 | 99.0 |
| 57 | 34.8 | 81.1 |
| 58 | 17.3 | 12.4 |
| 59 | 2.4 | 4.6 |
| 60 | 44.9 | 63.6 |
| 61 | 2.3 | 4.9 |
| 62 | 67.7 | 68.7 |
| 63 | 27.2 | 30.7 |

### Q. Example 17

Samples were prepared for an in vivo study. Four formulations were prepared for oral delivery, containing insulin-PEG conjugate (with 2 kDa PEG) with different permeation enhancers as detailed in Table 6.

TABLE 6

| | Sample | | Sample | | Sample | | Sample | |
|---|---|---|---|---|---|---|---|---|
| | 64 | | 65 | | 66 | | 67 | |
| Olive Oil | 1 | ml | 1 | ml | 1 | ml | 1 | ml |
| Insulin-PEG (2 kDa)/ | 4.8 | mg | 4.8 | mg | 4.8 | mg | | |
| α-cyclodextrin | | | | | | | | |
| POPE | 15 | mg | 3 | mg | | | | |
| DPC | 3 | mg | 3 | mg | 3 | mg | | |
| DSS | | | 3 | mg | 3 | mg | | |

| | | | |
|---|---|---|---|
| Treatment Dose | 75 | 75 | 75 |
| (Insulin equivalent | | | |
| IU/kg) | | | |

The peptide and oil for formulations 64-66 were sonicated until they appeared cloudy but homogenous. Then the remaining ingredients were added to each sample. The samples were sonicated again until they appeared cloudy but homogenous. The samples were stored overnight at room temperature. Normal SD Rats (8 rats/group) were fasted overnight before being dosed by oral gavage at insulin equivalent dosage of 75 IU/kg or olive oil alone (sample 67). Blood was collected at predose (−30 min) and post dose (10 min, 30 min, 1 h, 1.5 h, 2 h, 3 h, 4 h, 6 h, 8 h) for glucose measurements by a glucometer.

All rats given samples 64-66 had blood glucose increase approximately 50% within the first hour before falling back close to initial levels from 2-8 hours. A similar trend was observed in the vehicle group (sample 67) suggesting that samples 64-66 were not effective at reducing the blood glucose with these particular rats in this example.

### R. Example 18

Samples were prepared for an in vivo study. Four formulations were prepared for oral delivery, containing insulin-PEG conjugate (with 2 kDa PEG) with different carrier compounds, permeation enhancers, and mucoadhesive compounds as detailed in Table 7. The samples are similar to Example 17, with the exception that all of these samples contained PLGA as a mucoadhesive and the rats were given a higher dose (100 IU/kg).

TABLE 7

| | Sample | | Sample | | Sample | | Sample | |
|---|---|---|---|---|---|---|---|---|
| | 69 | | 70 | | 71 | | 72 | |
| Olive Oil | 1 | ml | 1 | ml | 1 | ml | 1 | ml |
| Insulin-PEG (2 kDa)/ | 6.4 | mg | 6.4 | mg | 6.4 | mg | | |
| α-cyclodextrin | | | | | | | | |
| POPE | 20 | mg | 20 | mg | | | | |
| DPC | 4 | mg | 4 | mg | 4 | mg | | |
| DSS | | | 4 | mg | 4 | mg | | |
| PLGA | 50 | mg | 50 | mg | 50 | mg | | |

| | | | |
|---|---|---|---|
| Treatment Dose | 100 | 100 | 100 |
| (Insulin equivalent | | | |
| IU/kg) | | | |

The peptide and oil for formulations 69-71 were sonicated until they appeared cloudy but homogenous. Then the remaining ingredients were added to each sample. The samples were sonicated again until they appeared cloudy but homogenous. The samples were stored overnight at room temperature. Normal SD Rats (8 rats/group) were fasted overnight before being dosed by oral gavage at insulin equivalent dosage of 100 IU/kg or olive oil alone (sample 72). Blood was collected at predose (−30 min) and post dose (10 min, 30 min, 1 h, 1.5 h, 2 h, 3 h, 4 h, 6 h, 8 h) for glucose measurements by a glucometer.

Rats given samples 69 had a glucose response similar to the control group (sample 72). One rat dosed with sample 70 had a significant reduction in blood glucose, with glucose levels between 23-39 mg/dL from 0.5 to 3 hours, 47 mg/dL at hour 4 and 44 mg/dL at hour 6; the glucose levels returned to baseline (˜75 mg/dL) at hour 8. The remaining rats in this group had a glucose response similar to the control group. One rat dosed with sample 71 had a significant reduction in blood glucose, with glucose levels of 64 mg/dL at 0.5 hours, between 35-40 mg/dL from 1 to 2 hours, between 53-57 mg/dL from 3 to 4 hours and back to near baseline by 6 hours. The remaining rats in this group had a glucose response similar to the control group. The presence of DSS and PLGA in samples 70 and 71 along with an increase in dosage from 75 IU/kg to 100 IU/kg contributed to further glucose reduction when compared to samples 64-66. This suggests that the presence of DSS and PLGA in the formulation contribute to drug absorption.

### S. Example 19

Samples were prepared for an in vivo study. Seven formulations were prepared for oral delivery, containing insulin-PEG conjugate (with 2 kDa PEG) with different permeation enhancers, mucoadhesive compounds, carrier compounds, and surfactants as detailed in Table 8.

TABLE 8

| | Sample | | Sample | | Sample | | Sample | | Sample | | Sample | | Sample | |
|---|---|---|---|---|---|---|---|---|---|---|---|---|---|---|
| | 73 | | 74 | | 75 | | 76 | | 77 | | 78 | | 79 | |
| Olive Oil | 0.9 | ml | 0.9 | ml | 0.9 | ml | 0.9 | ml | 0.9 | ml | 0.9 | ml | 0.9 | ml |
| DHA | 0.1 | ml | 0.1 | ml | 0.1 | ml | 0.1 | ml | 0.1 | ml | 0.1 | ml | 0.1 | ml |
| Insulin-PEG (2 kDa)/ | 6.4 | mg | 6.4 | mg | 6.4 | mg | | | 9 | mg | | | 6.4 | mg |
| α-cyclodextrin | | | | | | | | | | | | | | |
| Insulin-PEG (2 kDa) | | | | | | | | | | | 4.8 | mg | | |
| α-cyclodextrin | | | | | | | | | | | 8.0 | mg | | |
| POPE | 4 | mg | 4 | mg | 4 | mg | | | 4 | mg | | | 4 | mg |
| DPC | 3.2 | mg | 3.2 | mg | 3.2 | mg | | | 3.2 | mg | | | 16 | mg |
| DSS | 4 | mg | 4 | mg | 4 | mg | | | 4 | mg | | | 4 | mg |
| PLGA | 50 | mg | 50 | mg | 50 | mg | | | 50 | mg | | | 50 | mg |
| Span 80 | | | 10 | μl | | | | | | | | | | |
| Tween 80 | | | | | 10 | μl | | | | | | | | |

| | | | | | | |
|---|---|---|---|---|---|---|
| Treatment Dose | 100 | 100 | 100 | 100 | 100 | 100 |
| (Insulin equivalent | | | | | | |
| IU/kg) | | | | | | |

The inclusion complex in sample 77 was prepared differently than in samples 73-75 and 79. Insulin-PEG and 10 molar excess α-cyclodextrin were mixed in an aqueous solution and kept overnight at 4° C. to form a precipitate. The resulting precipitate was filtered to remove any soluble insulin-PEG and α-cyclodextrin prior to lyophilization and used in sample 77, this method of preparing the complex should result in less free cyclodextrin in the formulation. The peptide and oil for formulations 73-75 and 77-79 were sonicated until they appeared cloudy but homogenous. Then the remaining ingredients were added to each sample. The samples were sonicated again until they appeared cloudy but homogenous. The samples were stored overnight at room temperature. Normal SD Rats (8 rats/group) were fasted overnight before being dosed by oral gavage at insulin equivalent dosage of 100 IU/kg or olive oil and DHA alone (sample 76). Blood was collected at predose (−30 min) and post dose (10 min, 30 min, 1 h, 1.5 h, 2 h, 3 h, 4 h, 6 h, 8 h) for glucose measurements by a glucometer for samples 73-76 and post dose (10 min, 30 min, 1 h, 1.5 h, 2 h, 3 h, 4 h) for samples 77-79.

One rat dosed with sample 73 had a significant reduction in blood glucose, the baseline for this rat was 95 mg/dL, by 30 minutes the glucose dropped to 60 mg/dL, the glucose remained between 34-47 mg/dL from 1 to 3 hours, and was 65 mg/dL at 4 hours. All other rats that received samples 73-79 had blood glucose responses similar to the control. This suggests that sample 73 had the best absorption of the formulations tested.

### T. Example 20

Samples were prepared for an in vivo study. Four formulations were prepared for oral delivery, containing insulin-PEG conjugate (with 2 kDa PEG) or insulin, with different permeation enhancers, mucoadhesive compounds, carrier compounds, and protease inhibitors. Formulations were made with enteric coated capsules, which are designed to not dissolve until they reach the small intestine, or gelatin capsules, which should dissolve in the stomach. The details of the samples are shown in Table 9.

TABLE 9

| | Sample | | Sample | | Sample | | Sample | |
|---|---|---|---|---|---|---|---|---|
| | 82 | | 83 | | 84 | | 85 | |
| Olive Oil | 0.9 | ml | 0.9 | ml | 0.9 | ml | 0.9 | ml |
| DHA | 0.1 | ml | 0.1 | ml | 0.1 | ml | 0.1 | ml |
| Insulin-PEG (2 kDa)/α- | 15.3 | mg | 15.3 | mg | 15.3 | mg | 15.3 | mg |
| cyclodextrin | | | | | | | | |
| POPE | 5 | mg | 5 | mg | 5 | mg | 5 | mg |
| DPC | 4 | mg | 4 | mg | 4 | mg | 4 | mg |
| DSS | 5 | mg | 5 | mg | 5 | mg | 5 | mg |
| PLGA | 62.5 | mg | 62.5 | mg | 62.5 | mg | 62.5 | mg |
| SBTI | 62.5 | mg | 62.5 | mg | | | | |

| | | | | |
|---|---|---|---|---|
| Treatment Dose | 8 | 8 | 8 | 8 |
| (mg/animal) | | | | |
| Capsule Type | Enteric | Gelatin | Enteric | Gelatin |

The peptide and oil for formulations 82-85 were sonicated until they appeared cloudy but homogenous. Then the remaining ingredients were added to each sample. The samples were sonicated again until they appeared cloudy but homogenous. The samples were then added to capsules. In samples 82 and 83, SBTI was added to the capsules before the oil mixture was added. The samples were stored overnight at room temperature. Normal beagle dogs (6 dogs/group) were fasted overnight before being dosed with pills at an insulin equivalent dosage of 8 mg/dog. Blood was collected at predose (−30 min) and post dose (10 min, 30 min, 1 h, 1.5 h, 2 h, 3 h, 5 h, 7 h) for glucose measurements by a glucometer. Collected blood samples were also analyzed for insulin and c-peptide.

No significant glucose reduction was seen in the dogs dosed with sample 82. Two of six dogs given sample 83 had reductions in blood glucose of greater than 30%, with a maximum reduction at 0.5 hours, the glucose levels were back to baseline by 2 hours. No significant glucose reduction was seen in the other dogs in this group. No significant reductions in blood glucose were seen in dogs given samples 84, and 85. These results suggest that enteric coated capsules are not needed for successful delivery, as sample 83 was delivered in gelatin capsules. It also appears that the presence of the peptidase inhibitor SBTI enhanced the absorption by preventing digestion of the protein.

Serum insulin was measured by ELISA, increases in serum insulin levels were detected in the two dogs that had significantly reduced blood glucose, in addition some increase in insulin was detected in other samples. In sample 83, the maximum concentration of insulin was 3.7 ng/ml at 10 minutes and 6.4 ng/ml at 30 minutes for the two dogs that had a reduction in blood glucose. Insulin was detected in four dogs given sample 82, with a Cₘₐₓ between 1.0 ng/ml and 1.6 ng/ml occurring between 10 and 60 minutes. Insulin was detected in two dogs sample 84, with a Cₘₐₓ between 1.2 ng/ml and 1.3 ng/ml occurring between 60 and 90 minutes. Insulin was detected in two dogs given sample 85, with a Cₘₐₓ between 1.5 ng/ml and 1.8 ng/ml occurring between 10 and 30 minutes. Taken together, these results suggest that serum insulin levels greater than 1.8 ng/ml are needed to achieve glucose reduction.

In those dogs with reduced blood glucose, C-peptide levels also were suppressed. C-peptide is used as an indicator of endogenous insulin. Proinsulin is cleaved into insulin and C-peptide. If insulin is endogenous, then an equimolar amount of C-peptide is produced. When C-peptide levels drop, the animal is producing less insulin, which indicates that the exogenous insulin is taking the place of the endogenous insulin. The two dogs given sample 83 that had reduced glucose also had reductions in serum C-peptide from 64% to 92% of baseline levels. Taken together, the reductions in blood glucose and C-peptide with increases in serum insulin indicate that the reduction in blood glucose was caused by exogenous insulin.

### U. Example 21

Samples were prepared for an in vivo study. Four formulations were prepared for oral delivery, containing insulin-PEG conjugate (with 2 kDa PEG) or insulin, with different permeation enhancers, mucoadhesive compounds, carrier compounds, and protease inhibitors. Formulations were made with enteric coated capsules, which are designed to not dissolve until they reach the small intestine, or gelatin capsules, which should dissolve in the stomach. In addition, dosing of samples 86-89 was preceded by dosing with 200 mg of sodium bicarbonate in a separate gelatin capsule in an effort to raise the pH of the stomach. Raising the pH of the stomach should decrease pepsin activity, which has decreased activity above pH 2, potentially resulting in less degradation of the insulin in the stomach. In addition, increasing the pH of the stomach might help to improve insulin stability, since degradation can occur at low pH. The details of samples 86-89 are shown in Table 10.

TABLE 10

| | Sample | | Sample | | Sample | | Sample | |
|---|---|---|---|---|---|---|---|---|
| | 86 | | 87 | | 88 | | 89 | |
| Olive Oil | 0.9 | ml | 0.9 | ml | 0.9 | ml | | |
| DHA | 0.1 | ml | 0.1 | ml | 0.1 | ml | | |
| Insulin-PEG (2 kDa)/α- | 15.3 | mg | | | 15.3 | mg | 15.3 | mg |
| cyclodextrin | | | | | | | | |
| Insulin-PEG (2 kDa) | | | 6.8 | mg | | | | |
| α-cyclodextrin | | | 0.8 | mg | | | | |
| POPE | 5 | mg | 5 | mg | 5 | mg | 5 | mg |
| DPC | 4 | mg | 4 | mg | 4 | mg | 4 | mg |
| DSS | 5 | mg | 5 | mg | 5 | mg | 5 | mg |
| PLGA | 62.5 | mg | 62.5 | mg | 62.5 | mg | 62.5 | mg |
| SBTI | 62.5 | mg | 62.5 | mg | 62.5 | mg | 62.5 | mg |
| EDTA | | | | | 62.5 | mg | | |

| | | | | |
|---|---|---|---|---|
| Treatment Dose | 8 | 8 | 8 | 8 |
| (mg/animal) | | | | |
| Capsule Type | Gelatin | Gelatin | Gelatin | Gelatin |

The peptide and oil for formulations 86-88 were sonicated until they appeared cloudy but homogenous. Then the remaining ingredients were added to each sample. The samples were sonicated again until they appeared cloudy but homogenous. SBTI was added to the capsules prior to the oil mixture in samples 86 and 87, while SBTI and EDTA were added prior to the oil mixture in sample 88. Sample 89 was prepared by dissolving all components, except SBTI, in water. The samples were vortexed after each component was added until the mixture was homogenous. The mixture was then flash frozen and lyophilized, and added to capsules containing SBTI. Dosing of samples 86-89 was preceded by dosing with 200 mg of sodium bicarbonate in a separate gelatin capsule in an effort to raise the pH of the stomach. The samples were stored overnight at 4° C. Normal beagle dogs (6 dogs/group) were fasted overnight before being dosed with pills at an insulin equivalent dosage of 8 mg/dog. Blood was collected at predose (−30 min) and post dose (10 min, 30 min, 1 h, 1.5 h, 2 h, 3 h, 5 h, 7 h) for glucose measurements by a glucometer.

Of the dogs given sample 86, two had the greatest glucose reduction at 30 minutes (27% and 65% reduction), while one dog had the greatest glucose reduction at 1 h (39%). The remaining three dogs had glucose changes that were less than 15% of baseline values. The results are similar to those observed in sample 83, indicating that the presence of sodium bicarbonate did not significantly alter the drug absorption. Of the dogs given sample 87, four had the greatest glucose reduction at 30 minutes (37%, 41%, 51%, and 42%) with glucose levels returning to baseline after 30 additional minutes in three dogs and 1 hour in the fourth dog. The remaining two dogs had glucose changes that were less than 15% of baseline values. Of the dogs given sample 88, two had the greatest glucose reduction at 30 minutes (30% and 69% reduction), while one dog had the greatest glucose reduction at 1 h (53%). Glucose returned to baseline levels after 1 hour in two dogs and 3 hours in the third. The remaining three dogs had glucose changes that were less than 15% of baseline values. Of the dogs given sample 89, one had the greatest glucose reduction at 1 h (29%). The remaining five dogs had glucose changes that were less than 15% of baseline values, and therefore the carrier compound was observed to influence absorption.

### V. Example 22

Four samples were prepared with a peptide fragment of parathyroid hormone (PTH), consisting of amino acid residues 1-34, pegylated on the C-terminus (PTH-PEG). The PEG used for conjugation was either 2 kDa or 5 kDa, as specified below.

Sample 91: 1.68 mg PTH-PEG (2 kDa) and 0.2 mg α-cyclodextrin, 3 mg POPE, 2.4 mg DPC, 3 mg DSS, 50 mg PLGA, 0.8 mL olive oil.

Sample 92: 1.68 mg PTH-PEG (2 kDa) and 0.2 mg α-cyclodextrin, 3 mg POPE, 2.4 mg DPC, 3 mg DSS, 50 mg PLGA, 0.8 mL olive oil.

Sample 93: 2.5 mg PTH-PEG (5 kDa) and 0.2 mg α-cyclodextrin, 3 mg POPE, 2.4 mg DPC, 3 mg DSS, 50 mg PLGA, 0.8 mL olive oil.

Sample 94: 2.5 mg PTH-PEG (5 kDa) and 0.2 mg α-cyclodextrin, 3 mg POPE, 2.4 mg DPC, 3 mg DSS, 50 mg PLGA, 0.8 mL olive oil.

The peptide and oil for samples 91-94 were sonicated until they appeared cloudy but homogenous. Then the remaining ingredients were added to each sample. The samples were sonicated again until they appeared cloudy but homogenous. The samples were then added to 4 mL of simulated gastric fluid that did not include pepsin. The mixtures were inverted several times to mix.

The samples were run through High Performance Liquid Chromatography (HPLC) to determine if the PTH-PEG remained in the oil phase and how much went into the water phase. All samples had <40% that had left the oil phase and entered the water phase at 0.25 hours and very little PTH remaining in the oil at 3 hours. The results are shown in Table 11.

TABLE 11

| | % PTH-PEG in | % PTH-PEG in oil |
|---|---|---|
| Sample # | water phase 0.25 h | phase 3 h |
| 91 | 36% | 0.4% |
| 92 | 27% | 0.3% |
| 93 | 25% | 0% |
| 94 | 7% | 0% |

### W. Example 23

An in vivo study was performed to compare an oral PTH-PEG sample to a subcutaneous injection of PTH. A formulation was prepared containing PTH-PEG conjugate (amino acid residues 1-34 with 2 kDa PEG) for dosing by oral gavage in normal rats. For comparison, a sample was prepared with non-pegylated PTH (amino acid residues 1-34) for dosing by subcutaneous injection.

Sample 95: 2 mg PTH and 10 mL phosphate buffered saline pH 7, containing 0.01% Tween 80 (PBST).

Sample 96: 8.6 mg PTH-PEG and 1.3 mg α-cyclodextrin, 5 mg POPE, 4 mg DPC, 5 mg DSS, 62.5 mg SBTI, 0.9 mL olive oil, and 0.1 mL DHA.

The peptide for sample 95 was dissolved with PBST approximately 30 minutes prior to dosing and inverted several times to mix.

The peptide and oil for sample 96 was sonicated until it appeared cloudy but homogenous. Then the remaining ingredients were added to the sample and it was sonicated again until it appeared cloudy but homogenous. The sample was stored at 2-8° C. overnight (about 12 hours). SBTI was added to the formulation approximately 30 minutes before dosing.

Normal rats (5 rats/group) were fasted overnight. For sample 95, rats were dosed by subcutaneous injection at 0.2 mg PTH/mL/kg. For sample 96, rats were dosed by oral gavage at 15 mg PTH-PEG/kg (8.6 mg/mL). Blood was collected at predose (−30 min) and post dose (15 min, 1 h, 2 h, 4 h, 24 h). Serum samples were analyzed by ELISA for PTH and serum calcium concentration.

The rats dosed subcutaneously with sample 95 had maximum levels of PTH at 15 minutes ranging from 1,474 to 7,968 pg/mL. These levels rapidly declined and only two rats had measurable levels at 1 hour. The corresponding calcium levels of the rats given sample 95 reached maximum levels at 2 hours and ranged from 57.3 to 66.9 μg/mL. Of the five rats orally dosed with sample 96, only one had measurable PTH levels, with a Cₘₐₓ of 178,585 pg/mL at 15 minutes. The PTH levels for this rat slowly declined, but was still measurable at 24 hours (1,813 pg/mL). The serum calcium concentration, for this rat, reached maximum levels at 1 hour (80.2 μg/mL), and the levels were back to baseline (˜50 μg/mL) between 2 and 4 hours. The serum calcium concentration for the remaining 4 rats reached maximum levels at 1 hour, with the values ranging from 56.4 to 73.5 μg/mL. The average calcium levels remained elevated at the 2 hour timepoint and were near baseline by 4 hours. This experiment demonstrated that our technology can be used for both basic and acidic protein drugs. Insulin is an acidic protein (pI of 5.5) and PTH is basic (pI 8.0), therefore compositions can include both basic and acidic proteins.

### X. Example 24

Four samples were prepared with glucagon-like peptide-1 (GLP-1), GLP-PEG conjugate (with 2 kDa PEG or 5 kDa PEG), or insulin.

Sample 97: 0.72 mg GLP-1 and 0.2 mg α-cyclodextrin, 3 mg POPE, 2.4 mg DPC, 3 mg DSS, 50 mg PLGA, 0.8 mL olive oil.

Sample 98: 1.25 mg GLP-1-PEG (2 kDa) and 0.2 mg α-cyclodextrin, 3 mg POPE, 2.4 mg DPC, 3 mg DSS, 50 mg PLGA, 0.8 mL olive oil.

Sample 99: 1.84 mg GLP-1-PEG (5 kDa) and 0.2 mg α-cyclodextrin, 3 mg POPE, 2.4 mg DPC, 3 mg DSS, 50 mg PLGA, 0.8 mL olive oil.

Sample 100: 1.36 mg insulin (5 kDa) and 0.2 mg α-cyclodextrin, 3 mg POPE, 2.4 mg DPC, 3 mg DSS, 50 mg PLGA, 0.8 mL olive oil.

The peptide and oil for samples 97-100 were sonicated until they appeared cloudy but homogenous. Then the remaining ingredients were added to each sample. The samples were sonicated again until they appeared cloudy but homogenous. sample 98 was noticeably cloudier than the other samples. The samples were then added to 4 mL of simulated gastric fluid that did not include pepsin. The mixtures were inverted several times to mix.

The samples were run through High Performance Liquid Chromatography (HPLC) to determine if the protein or pegylated protein remained in the oil phase and how much went into the water phase. In samples 97-100, protein was not quantifiable in the water phase. Some protein did remain in the oil phase after 3 hours in samples 97, 98, and 100 as shown in Table 12. More un-PEGylated GLP-1 (sample 97) was protected in the oil phase than the GLP-1 with the 2 kDa PEG (sample 98), or the GLP-1 with the 5 kDa PEG (sample 99), suggesting that PEG molecular weight contributes to the GLP-1 partitioning in the oil.

TABLE 12

| | % API in water | % API in oil phase |
|---|---|---|
| Sample # | phase 0.25 h | 3 h |
| 97 | Not quantifiable | 12.3% |
| 98 | Not quantifiable | 0.4% |
| 99 | Not quantifiable | 0% |
| 100 | Not quantifiable | 6.0% |

### Y. Example 25

Four samples were prepared with the oil soluble small molecule esomeprazole magnesium hydrate.

Sample 101: 1.94 mg esomeprazole magnesium hydrate, 3 mg POPE, 2.4 mg DPC, 3 mg DSS, 50 mg PLGA, 0.8 mL olive oil.

Sample 102: 1.11 mg esomeprazole magnesium hydrate, 1 mg α-cyclodextrin, 3 mg POPE, 2.4 mg DPC, 3 mg DSS, 50 mg PLGA, 0.8 mL olive oil.

Sample 103: 33.6 mg esomeprazole magnesium hydrate β-cyclodextrin inclusion complex, 3 mg POPE, 2.4 mg DPC, 3 mg DSS, 50 mg PLGA, 0.8 mL olive oil.

Sample 104: 33.9 mg esomeprazole magnesium hydrate γ-cyclodextrin inclusion complex, 3 mg POPE, 2.4 mg DPC, 3 mg DSS, 50 mg PLGA, 0.8 mL olive oil.

In samples 103 and 104, inclusion complexes were formed by combining esomeprazole magnesium hydrate with a 10 molar excess of β or γcyclodextrin in aqueous solution. After overnight incubation at 4° C., a white precipitate formed, which was then flash frozen and lyophilized.

The esomeprazole magnesium hydrate and oil for samples 101-104 were sonicated until they appeared cloudy but homogenous. Then the remaining ingredients were added to each sample. The samples were sonicated again until they appeared cloudy but homogenous. The samples were then added to 4 mL of a solution of 50% acetonitrile and 50% PBS that did not include pepsin. The mixtures were inverted several times to mix.

The samples were run through High Performance Liquid Chromatography (HPLC) to determine if the esomeprazole magnesium hydrate remained in the oil phase and how much went into the water phase. The results are shown in Table 13.

TABLE 13

| | % esomeprazole | | |
|---|---|---|---|
| | magnesium | | |
| | hydrate | % esomeprazole | % esomeprazole |
| | in water phase | magnesium hydrate | magnesium hydrate |
| Sample # | 0.25 h | in water phase 1 h | in water phase 3 h |
| 101 | 24.0 | 38.3 | 40.5 |
| 102 | 20.1 | 28.2 | 31.8 |
| 103 | 9.7 | 14.7 | 18.2 |
| 104 | 8.8 | 14.5 | 19.8 |

The amount of esomeprazole magnesium hydrate that entered the water phase was reduced when α-cyclodextrin was added to the oil mixture, as in sample 102. The amount of esomeprazole magnesium hydrate that entered the water phase was further reduced when an inclusion complex with β or γcyclodextrin was added to the oil mixture, as in samples 103 and 104.

### Z. Example 26

Two samples were prepared with the water soluble small molecule ceftriaxone sodium.

Sample 105: 2.15 mg ceftriaxone sodium, 3 mg POPE, 2.4 mg DPC, 3 mg DSS, 50 mg PLGA, 0.8 mL olive oil.

Sample 106: 1.85 mg ceftriaxone sodium, 1 mg α-cyclodextrin, 3 mg POPE, 2.4 mg DPC, 3 mg DSS, 50 mg PLGA, 0.8 mL olive oil.

| | % ceftriaxone | % ceftriaxone | % ceftriaxone |
|---|---|---|---|
| | sodium in water | sodium in water | sodium in water |
| Sample # | phase 0.25 h | phase 1 h | phase 3 h |
| 105 | 26.6 | 38.9 | 45.8 |
| 106 | 31.0 | 38.7 | 48.6 |

The ceftriaxone sodium and oil for samples 105-106 were sonicated until they appeared cloudy but homogenous. Then the remaining ingredients were added to each sample. The samples were sonicated again until they appeared cloudy but homogenous. The samples were then added to 4 mL of a solution simulated gastric fluid. The mixtures were inverted several times to mix.

The absorbance of the simulated gastric fluid at 300 nm was used to determine if the ceftriaxone sodium went into the water phase.

The results indicate that the ceftriaxone sodium slowly entered the water phase in samples 105 and 106, with only 50% in the water phase at 3 hours, suggesting that the other 50% remains in the oil phase.

Examples 1-26 are repeated with human growth hormone, glucagon-like peptide-1, parathyroid hormone, a fragment of parathyroid hormone, enfuvirtide, and octreotide in place of the active pharmaceutical ingredient.

All patents, patent publications, patent applications, journal articles, books, technical references, and the like discussed in the instant disclosure are incorporated herein by reference in their entirety for all purposes.

In the preceding description, for the purposes of explanation, numerous details have been set forth in order to provide an understanding of various embodiments of the present technology. It will be apparent to one skilled in the art, however, that certain embodiments may be practiced without some of these details, or with additional details.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Additionally, details of any specific embodiment may not always be present in variations of that embodiment or may be added to other embodiments.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a method” includes a plurality of such methods and reference to “the tissue” includes reference to one or more tissues and equivalents thereof known to those skilled in the art, and so forth. The invention has now been described in detail for the purposes of clarity and understanding. However, it will be appreciated that certain changes and modifications may be practice within the scope of the appended claims.

1. A composition for oral drug delivery, the composition comprising:
a physiologically active substance;
a carrier compound;
a mucoadhesive compound; and
a permeation enhancer.

2. The composition of claim 1, wherein the physiologically active substance comprises insulin, human growth hormone, glucagon-like peptide-1, parathyroid hormone, a fragment of parathyroid hormone, enfuvirtide, or octreotide.

3. The composition of claim 1, wherein the physiologically active substance comprises insulin or an insulin-PEG conjugate.

4. The composition of claim 3, wherein:
the physiologically active substance comprises the insulin-PEG conjugate, and
the insulin-PEG conjugate comprises a PEG with a molecular weight in a range from 2 kDa to 5 kDa.

5. The composition of claim 1, wherein the physiologically active substance comprises an insulin analog, homolog, or derivative.

6. The composition of claim 1, wherein the physiologically active substance comprises GLP-1 or a GLP-1-PEG conjugate.

7. The composition of claim 6, wherein:
the physiologically active substance comprises the GLP-1-PEG conjugate, and
the GLP-1-PEG conjugate comprises a PEG with a molecular weight in a range from 2 kDa to 5 kDa.

8. The composition of claim 1, wherein the physiologically active substance comprises a GLP-1 analog, homolog, or derivative.

9. The composition of claim 1, wherein the carrier compound is water insoluble.

10. The composition of claim 1, wherein the carrier compound comprises an amphipathic and water-immiscible compound.

11. The composition of claim 1, wherein the carrier compound comprises fish oil, esterified triglycerides, omega fatty acids, olive oil, orange oil, krill oil, lemon oil, safflower oil, castor oil, hydrogenated oils, or mixtures thereof.

12. The composition of claim 1, wherein the mucoadhesive compound comprises a cyclodextrin, a starch, a poly(d,l-lactide-co-glycolide), a caprolactone, or a food additive.

13. The composition of claim 1, wherein the permeation enhancer comprises a positively charged molecule, a negatively charged molecule, or a zwitterionic molecule.

14. The composition of claim 1, wherein the permeation enhancer comprises an amphiphilic molecule.

15. The composition of claim 1, wherein the permeation enhancer comprises an alkyl glucoside, an alkyl choline, an acyl choline, a bile salt, a phospholipid, or a sphingolipid.

16. The composition of claim 1, wherein the permeation enhancer comprises dodecylphosphocholine or sodium dodecyl sulfate.

17. The composition of claim 1, further comprising a capsule encapsulating the physiologically active substance, the carrier compound, the mucoadhesive compound, and the permeation enhancer, wherein the capsule is configured to degrade in a stomach.

18. The composition of claim 1, wherein the composition does not comprise an enteric coating and does not comprise a peptidase inhibitor.

19. The composition of claim 1, further comprising a hydrophobic anion of an organic acid.

20. The composition of claim 19, wherein the organic acid comprises pamoic acid, docusate, furoic acid, or mixtures thereof.

21. The composition of claim 19, wherein the hydrophobic anion of the organic acid comprises a fatty acid anion, a phospholipid anion, a polystyrene sulfonate anion, or mixtures thereof.

22. The composition of claim 1, wherein:
the mucoadhesive compound comprises a cyclodextrin, and
the physiologically active substance and the mucoadhesive compound form an inclusion complex in the cyclodextrin.

23. The composition of claim 1, further comprising a biodegradable polymer, wherein the biodegradable polymer forms a particle comprising the physiologically active substance.

24. The composition of claim 23, wherein the biodegradable polymer comprises poly(d,l-lactide-co-glycolide).

25. The composition of claim 1, further comprising a pH modifier.

26. The composition of claim 1, further comprising a peptidase inhibitor.

27.-52. (canceled)
